(19) United States
(12) Patent Application Publication
(10) Pub. No.: US 2015/0377887 A1
(43) Pub. Date: Dec. 31, 2015

(54) IN SITU AFFINITY MATURATION OF ANTIBODIES

(71) Applicants: Yu-cheng SU, Taipei (TW); Steve R. ROFFLER, Taipei (TW); ACADEMIA SINICA, Taipei (TW)

(72) Inventors: Yu-Cheng Su, Taipei (TW); Steve R. ROFFLER, Taipei (TW)

(21) Appl. No.: 14/769,294

(22) PCT Filed: Feb. 27, 2014
(86) PCT No.: PCT/IB2014/000784

§ 371 Date: Aug. 20, 2015

Related U.S. Application Data
(60) Provisional application No. 61/769,856, filed on Feb. 27, 2013.

(51) Int. Cl. G01N 33/577; C07K 16/40; C07K 16/44; G01N 33/68; G01N 33/50

(52) U.S. Cl. CPC G01N 33/577; G01N 33/6854; G01N 33/5008; C07K 16/44; C07K 16/40; C07K 2317/14

(57) ABSTRACT

This disclosure relates to a method to mimicking the germinal center reaction to generate antibodies with altered binding properties or increased affinity directly in hybridomas. To allow convenient and rapid affinity maturation of antibodies, a controllable activation-induced cytidine deaminase (AID) expression system to induce somatic hypermutation in hybridomas is developed. Selection of high affinity antibodies is achieved by fluorescence-activated cell sorting of hybridomas that preferentially bind fluorescence-labeled antigens. The disclosure also relates to de novo generation of hybridomas with tunable antibody affinity by generating myeloma fusion partners with controllable AID expression.

### CROSS-REFERENCE TO RELATED APPLICATION

This application claims benefit of U.S. Provisional Application No. 61/769,856, filed Feb. 27, 2013, which is incorporated by reference herein in its entirety.

### BACKGROUND OF THE INVENTION

Monoclonal antibodies are widely used for assays and therapy. Antibody therapeutics represents a multi-billion dollar business. The antigen-binding affinity of antibodies is important for both effective therapeutic and diagnostic applications^{1, 2}

Three major technologies have been developed to create and select mAbs, including generating hybridomas from immunized animals,³⁻⁶ selection of recombinant antibodies from libraries displayed in microorganisms,⁷⁻¹⁰ and immortalization of B lymphocytes isolated from human subjects.¹¹⁻¹³ Although immunization of transgenic animals provide a powerful method to generate antibodies against many antigens, it is difficult to obtain high affinity antibodies to T-cell-independent antigens such as tumor-associated carbohydrates^{14, 15} and PEG.¹⁶ In addition, antibodies selected from microbial display libraries may not only show suboptimal binding affinities for therapeutic and diagnostic applications^{1, 2} but also lack proper glycosylation. Furthermore, it is not feasible to immunize humans with many antigens, restricting immortalized human B lymphocytes from patient donors to infectious diseases. Accordingly, the development of a universal antibody screening system that combined with continuous mutation of antibody genes and functional expression of glycosylated antibodies in mammalian cell-based expression system may offer a tool for isolation of high affinity antibodies from small repertoire libraries including the library from low immunogenic T-cell-independent hapten immunization.

Antibodies with higher affinity may increase the therapeutic index by allowing low-dose administration of antibodies to elicit similar therapeutic effects with lower dose-related toxicity. Thus, many mutagenesis and engineering strategies have been developed to enhance antibody binding affinity, including complementarity determining region (CDR) mutagenesis,¹⁷⁻²¹ error-prone PCR and DNA shuffling.²²⁻²⁵ Although these approaches commonly result in improvements in affinity,⁹ the cloning of antibody genes and subsequent affinity maturation is technically challenging, time consuming and expensive. Furthermore, phage libraries can lead to antibodies that bind only via the heavy chain variable region but not light chain variable region.²⁶ By contrast, more natural somatic hypermutation can promote generation of novel antibodies that are difficult to obtain by molecular cloning and phage libraries.^{26, 27} We have invented a novel antibody affinity enhancement technique that closely resembles the natural affinity maturation process in germinal center B cells.

In the mammalian immune system, pre-B cells in the bone marrow undergo V-D-J recombination of heavy and light chain genes to generate the primary B-cell receptor (BCR, surface immunoglobulin) repertoire.²⁸ Contact of B cells with cognate antigen in germinal centers leads to induction of activation-induced cytidine deaminase (AID) dependent deamination of cytidine followed by error-prone DNA repair that introduces point mutations in the V regions of immunoglobulin (Ig) genes in centroblast B cells.²⁹⁻³² Selection of beneficial mutations during affinity maturation leads to the generation of B cells that elaborate high affinity IgG, IgA and IgE antibodies.³³ The expression of AID is down-regulated in differentiated plasma cells³⁴⁻³⁶ and hybridomas.³⁷ Artificial over-expression of AID in hybridomas can induce mutations in the V region of endogenous antibody genes.³⁸

Here, we mimicked the germinal center reaction to generate antibodies with altered properties or increased affinity directly in hybridomas. The affinity depends on the application and antigen, but typically a range of dissociation constants K_{D}=10⁻⁸ to 10⁻¹⁰ M is appropriate. K_{D} can be measured by surface plasmon resonance in a Biacore instrument, by flow cytometry or by ELISA. To allow convenient and rapid affinity maturation of antibodies, we developed a controllable AID expression system to induce somatic hypermutation in hybridomas. Selection of high affinity antibodies can be achieved by fluorescence-activated cell sorting of hybridomas that preferentially bind fluorescence-labeled antigens (FIG. 1). This approach represents a general and powerful approach to antibody affinity maturation. The general process is as follows: 1) Lentiviral particles expressing inducible or constitutive AID are infected into target hybridomas and selected in puromycin. 2) Expression of AID induces somatic hypermutation of the antibody variable region genes, generating a population of hybridomas with a distribution of affinities. 3) Limiting amounts of fluorescence-labeled antigen (PEG in our case) is added and hybridomas are sorted on a fluorescence-activated cell sorter to collect hybridomas expressing membrane-anchored high affinity antibodies. The process is repeated until hybridomas expressing (secreting) sufficiently high affinity antibodies are isolated. Hypermutation is terminated by removal of doxycycline or by CRE recombination to remove the AID cassette. In addition, we further extended this technology to de novo generation of hybridomas with tunable antibody affinity by generating myeloma fusion partners with controllable AID expression.

The advantages of mimicking the germinal center reaction in vitro include the ability to alter or enhance antigen-binding without cloning antibody genes, widespread applicability to any hybridoma, and ability to harness the natural somatic hypermutation process to obtain high affinity antibodies against “difficult” antigens such as poly(ethylene glycol) or carbohydrates that can be difficult to obtain from phage libraries. Any existing hybridoma can be transduced with AID to initiate somatic hypermutations in the antibody variable region genes. Fluorescence-activated cell sorting can then be used to efficiently identify hybridomas expressing high affinity antibody variants. Because surface expression requires proper folding of the immunoglobulin gene, this selection process also simultaneously identifies antibodies that are properly folded and stably expressed. Furthermore, this technology can be used to confer the ability of any newly formed hybridoma to undergo somatic hypermutation and affinity maturation by following standard hybridoma techniques using myeloma cell lines (fusion partner) that express AID in a controllable manner. Thus, affinity maturation can be carried out without the need to clone antibody genes or perform any additional work beyond the widely used hybridoma technique to generate monoclonal antibodies. This technology is also compatible with hybridomas that secrete fully human antibodies, such as those generated from human B cells or B cell obtained from transgenic human antibody mice (i.e., UltiMab platform and Xenomouse). This is accomplished by immunization of these mice and generation of hybridomas using the AID-expressing myeloma cells to generate hybridomas. In all these applications, somatic hypermutation can be easily terminated after the desired affinity is achieved by simply removing doxycycline or transfecting cells with CRE recombinase. We also anticipate that the technology we describe here may lead to the generation of less immunogenic human antibodies because the mutations are “naturally” introduced by a process that closely mimics the natural affinity maturation process in vivo.

### BRIEF DESCRIPTION OF THE DRAWINGS

FIG. 1 is a representation of the affinity maturation strategy.

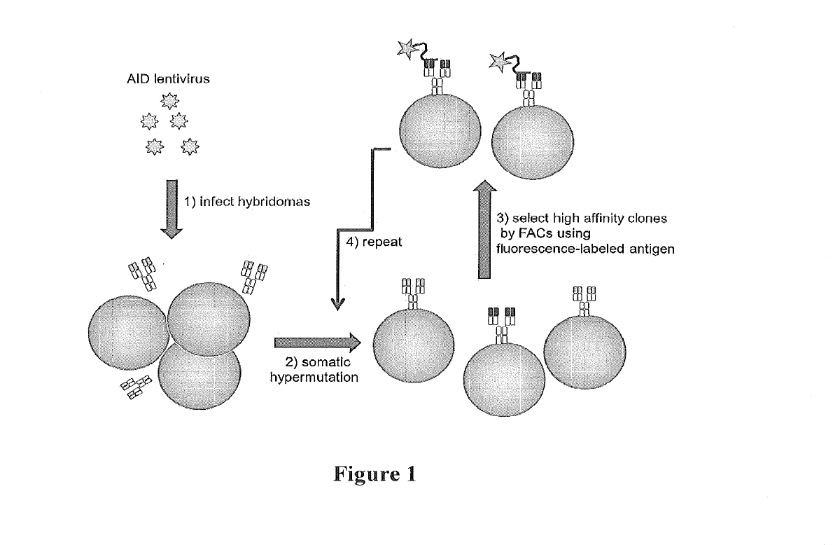

FIG. 2 is a representation of vectors for the controllable expression of AID

FIG. 3 shows in graphic form hybridomas expressing immunoglobulin on their surface.

FIG. 4 shows specific immunofluorescence staining of hybridomas.

FIG. 5 shows functional antigen-binding of hybridomas.

FIG. 6 is a schematic representation of loxp flanked mAID construct.

FIG. 7 is a representation of a flow cytometric analysis of Cre excision of the AID cassette using fluorescence-activated cell sorting analysis.

FIG. 8 is a representation of a flow cytometric analysis of Cre excision of the AID cassette.

FIG. 9 is a representation of TetOn-mAID and the strategy to detect AID activity.

FIG. 10 shows flow cytometry histograms od 3T3/Tet-On-mAID fibroblasts induced with varying concentrations of doxycycline.

FIG. 11 shows inducible expression of AID.

FIG. 12 is a graph showing mAID is functional.

FIG. 13 is a graphic analysis of the PEG-binding by 3.3/IoxP-mAID cells.

FIG. 14 shows amino acid sequences and alignment of immunoglobulin V gene sequences from 3.3/IoxP-mAID subclones.

FIG. 15 shows temperature-dependent binding of the anti-PEG antibody variants.

FIG. 16 shows the thermostability of the anti-PEG antibody variants.

FIG. 17 shows the expression of eGFP in FO and FO-AID myeloma cells.

FIG. 18 is a graphic representation of the expression of eGFP in hybridomas.

FIG. 19 is a graphic representation of the expression of mAID in hybridomas.

FIG. 20 shows the expression of mAID in FO-AID cells and hybridomas.

FIG. 21 is a graphic representation of enzyme-linked immunosorbent assay of 4 selected anti-human beta-glucuronidase hybridoma clones generated from the FO-AID myeloma fusion partner.

FIG. 22 shows flow cytometry antigen-binding analyses of 1-3-4 and 1-3-4-S5 hybridomas.

FIG. 23 shows affinity maturation of AGP4 anti-PEG IgM monoclonal antibody-round I.

FIG. 24 shows affinity maturation of AGP4 anti-PEG IgM monoclonal antibody-round II.

FIG. 25 shows affinity maturation of AGP4 anti-PEG IgM monoclonal antibody-round III.

FIG. 26 shows affinity maturation of AGP4 anti-PEG IgM monoclonal antibody-comparison of parental and round II and round III AFP4 hybridoma cell staining.

FIG. 27 shows affinity maturation and class switch of AGP4 anti-PEG IgM monoclonal antibody.

FIG. 28 shows class switch of AGP4 anti-PEG IgM monoclonal antibody to IgG.

FIG. 29 shows that class switched AGP4 anti-PEG IgG monoclonal antibodies retain antigen binding.

FIG. 30 shows affinity maturation of AGP4 class-switched IgG monoclonal antibody-round IV.

FIG. 31 shows class switching of anti-melanoma cell 3D8 IgM.

FIG. 32 shows that AID can be introduced into new hybridoma cells without virus infection.

### DETAILED DESCRIPTION OF THE INVENTION

### Examples

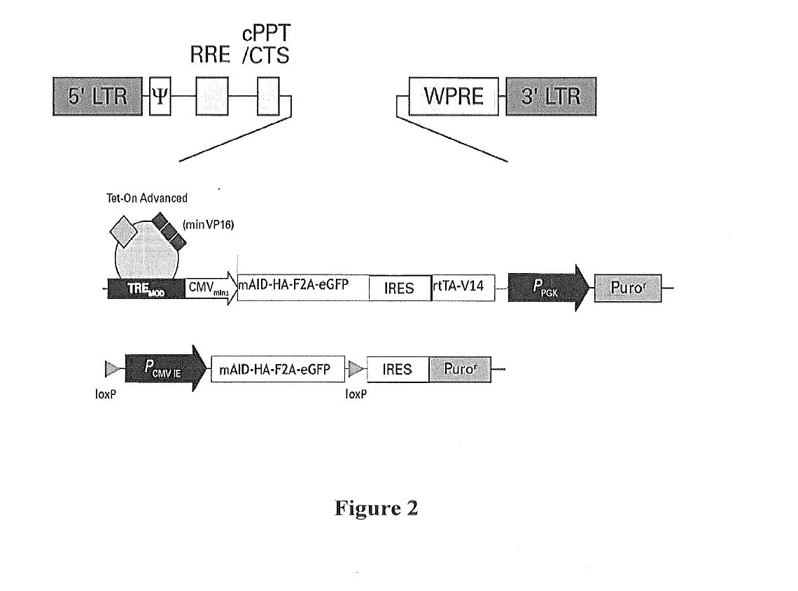

In the first embodiment of this invention, antibody-secreting hybridomas were transduced with an AID gene to induce somatic hypermutations in the antibody variable region genes. Expression of AID is controllable so somatic hypermutation can be terminated when antibodies with sufficient antigen-binding activity are obtained. We generated two vectors to allow controllable expression of AID. The expression of both AID and GFP are inducible in the first lentiviral vector whereas AID and GFP expression are constitutive in the second lentiviral vector, but can be stopped by transfection of the cells with a CRE recombinase to remove the expression cassette (FIG. 2). The symbols used in the figure mean the following: A, Vectors to express AID (activation-induced cytidine deaminase) in hybridomas in a controllable manner. HA epitope-tagged AID is expressed with GFP as a marker by introduction of the F2A sequence for ribosome skipping. In the first vector, AID expression is inducible by addition of doxycycline. In the second vector, AID is constitutively expressed, but can be removed from hybridomas by CRE transfection. Stable transfectants can be selected in puromycin. WPRE: woodchuck hepatitis virus posttranscriptional regulatory element. cPPT/CTS: central polypurine tract/central termination sequence. RRE: Rev response element.

### Characterization of Surface Immunoglobulin in Hybridomas

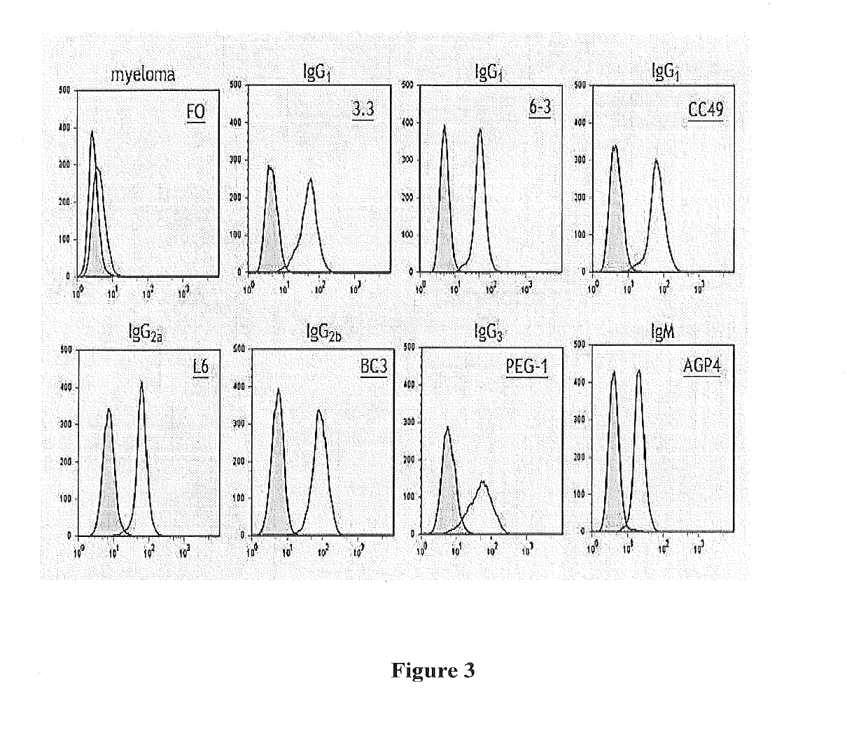

A key feature of the present invention is the ability to conveniently and rapidly identify hybridomas that secrete antibodies with higher affinity, altered specificity or enhanced properties. The surface expression of Ig on hybridomas was examined by staining with live cells with FITC-conjugated goat anti-mouse Ig antibody. All tested hybridomas, including those that secreted IgG₁, IgG₂ₐ, IgG_{2b}, IgG₃ and IgM antibodies displayed moderate to high level of surface Ig when compared with hybridoma fusion partner cell line, FO myeloma (FIG. 3). FO myeloma and hybridomas expressing different subclasses of monoclonal antibodies were immunofluorescence stained with anti-immunoglobulin F(ab′)2 antibody and analyzed for surface fluorescence in a fluorescence-activated cell sorter. Hybridomas secreting monoclonal antibodies with a range of subclasses and antigen-binding specificities displayed membrane-anchored antibody on their surface (open curves). Because the same antibody is secreted and expressed on the surface of individual hybridomas, surface immunoglobulin can be assessed to identify hybridomas secreting antibodies with the desired properties.

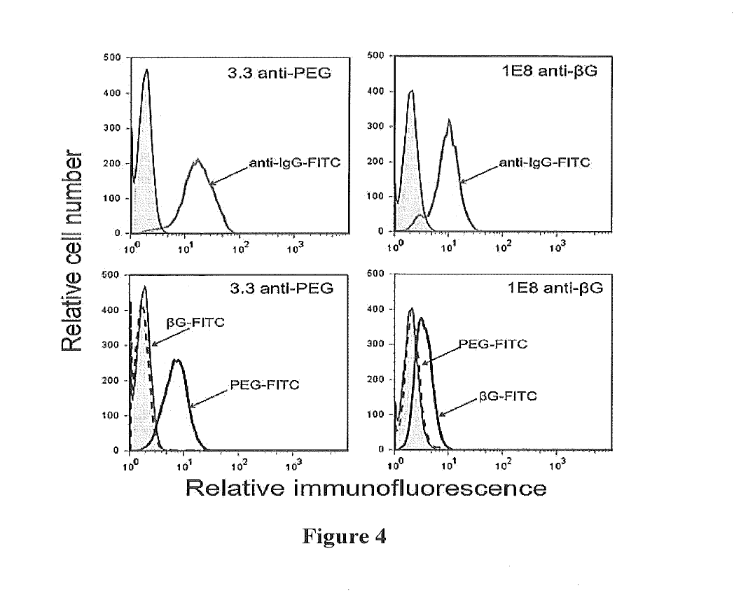

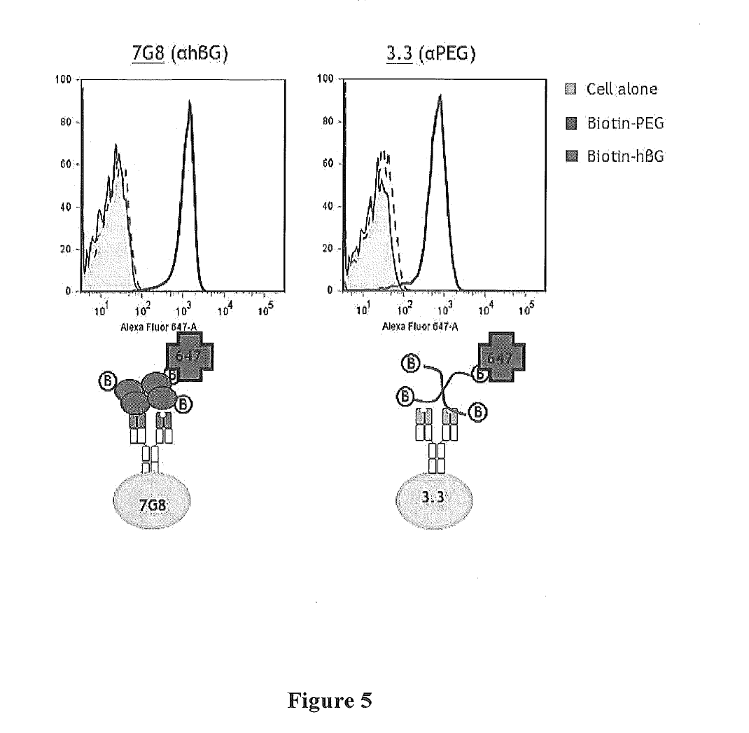

We further verified that surface immunoglobulin on hybridomas displayed the appropriate antigen-binding specificity as demonstrated by the binding of fluorescence-labeled antigens (FIG. 4). 3.3 and 1E8 hybridomas secreting antibodies against PEG and beta-glucuronidase (βG), respectively, were stained for surface immunoglobulin (top panels) or with FITC-labeled antigens (bottom panels). The 3.3 hybridoma bound PEG-F ITC but not βG-FITC whereas the 1E8 hybridoma bound to βG-FITC but not PEG-FITC. Thus, the 3.3 anti-PEG hybridoma bound FITC-labeled PEG molecules but not to control FITC-labeled beta-glucuronidase whereas the 1E8 anti-beta-glucuronidase hybridoma displayed the opposite specificity. Along the same lines, 3.3 anti-PEG and 7G8 anti-human beta-glucuronidase hybridoma cells were labeled with biotinylated polyethylene glycol (biotin-PEG) or human beta-glucuronidase (biotin-hβG), followed by Alexa647-conjugated streptavidin. FIG. 5 shows that 3.3 cells could specifically bind biotin-PEG but not biotin-human beta-glucuronidase, whereas 7G8 cells could bind biotin-human beta-glucuronidase but not biotin-PEG. Surface Ig on 3.3 anti-PEG hybridoma cells and 7G8 anti-human beta-glucuronidase hybridoma cells were analyzed on a FACScalibur flow cytometer using biotinylated PEG or human beta-glucuronidase (hβG) antigens then followed by Alexa647-conjugated strepavidin. We conclude that hybridomas express functional surface Ig.

Stoppable-Expression of mAID in Hybridoma Cells

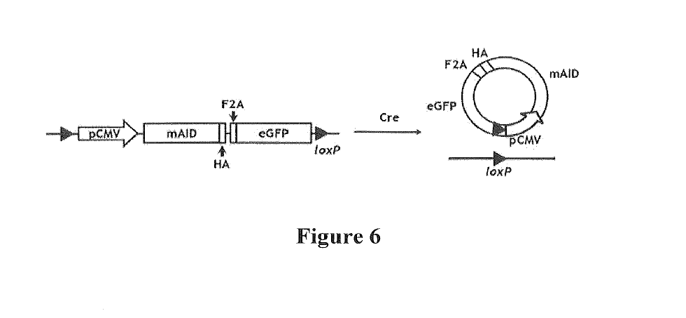

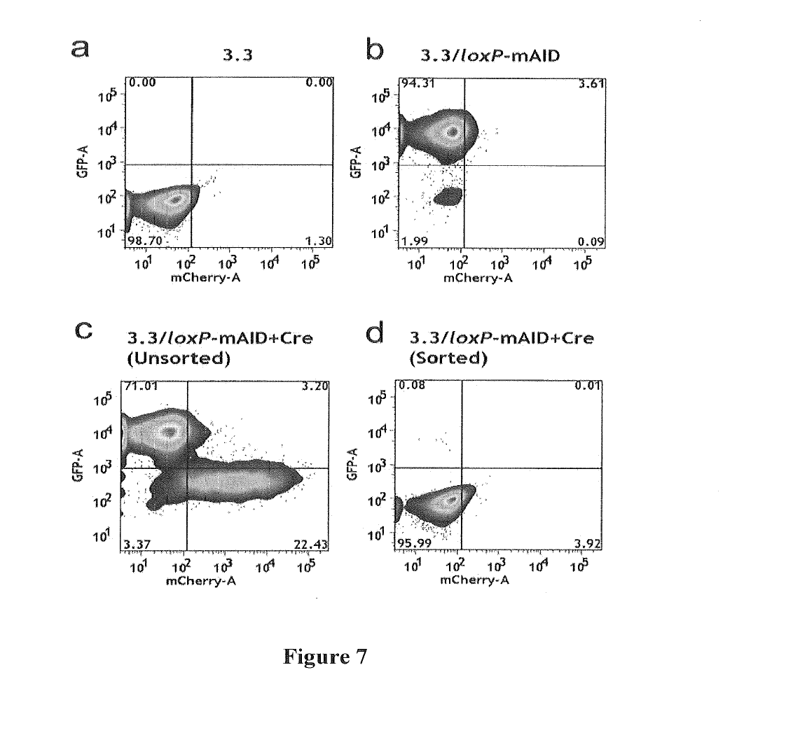

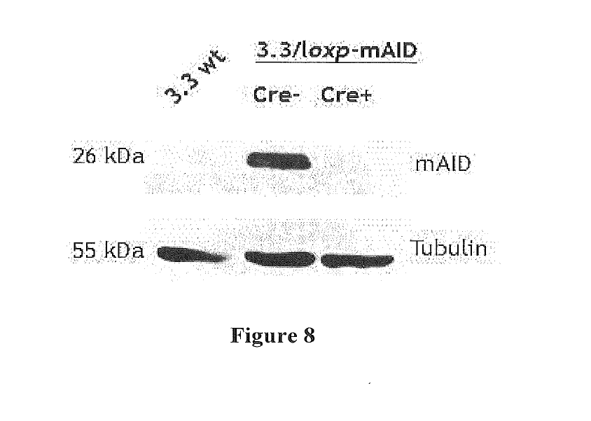

To generate stable hybridomas that express mAID in a controllable manner for in vitro somatic hypermutation, we transduced a IoxP flanked constitutive expression cassette (IoxP-CMV-mAID-F2A-eGFP-IoxP) (FIG. 6) to 3.3 hybridoma cells (3.3/IoxP-mAID). The IoxP flanked mAID is comprised of a CMV immediate early promoter, the mouse activation induced deaminase (mAID) gene, a HA epitope, a furin/2A peptide (F2A) bicistronic expression linker and an eGFP reporter gene. The mAID cassette is flanked by two IoxP motifs before the CMV promoter and after the eGFP DNA fragments for later Cre recombinase-mediated gene excision. FIGS. 7a-7d show fluorescence-activated cell sorting analysis of green fluorescence, representing AID expression (y-axis) and red fluorescence of mCherry, representing Cre expression (x-axis) in 3.3 cells (a), 3.3/IoxP-mAID cells that stably expressed IoxP flanked mAID (b), 3.3/IoxP-mAID cells that were transiently transfected with a pLM-mCherry-P2A-Cre plasmid by DNA electroporation (c), and 3.3/IoxP-mAID cells that were transiently transfected with a pLM-mCherry-P2A-Cre plasmid after sorting for eGFP-negative cells. FACs analysis showed that 3.3/IoxP-mAID (FIG. 7b) but not parental 3.3 (FIG. 7a) cells expressed green fluorescence corresponding to the AID reporter GFP protein. To test whether the expression of mAID could be stopped, a pLM-mCherry-P2A-Cre plasmid was transferred into 3.3/IoxP-mAID cells by DNA electroporation. About 23% of the cells expressed Cre recombinase after DNA electroporation as visualized by mCherry expression (FIG. 7c). These cells were also eGFP-negative (FIG. 7c), demonstrating that AID was eliminated from the cells that expressed CRE. The eGFP-negative cells were isolated on a FACSAria cell sorter (FIG. 7d) to confirm Cre-dependent deletion of the mAID gene. Immunoblotting of total cell lysates prepared from 3.3 cells, 3.3/IoxP-mAID cells or sorted Cre-treating 3.3/IoxP-mAID cells showed a band with the expected size of 26 kDa in 3.3/IoxP-mAID cells but not in 3.3 and Cre-treated 3.3/IoxP-mAID cells (FIG. 8). Cell lysates prepared from 3.3 hybridoma cells, 3.3/IoxP-mAID hybridoma cells or Cre-treated 3.3/IoxP-mAID hybridoma cells (sorted) were immunoblotted for the HA epitope tag on AID or tubulin as a cell loading control. These results demonstrate that mAID could be stably expressed and conditionally silenced by Cre recombinase mediated gene deletion in 3.3 hybridomas.

### Induction of SHM Against an Artificial Substrate

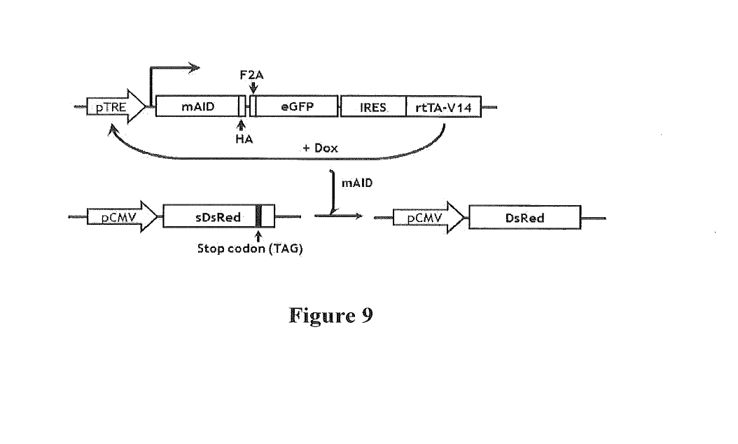

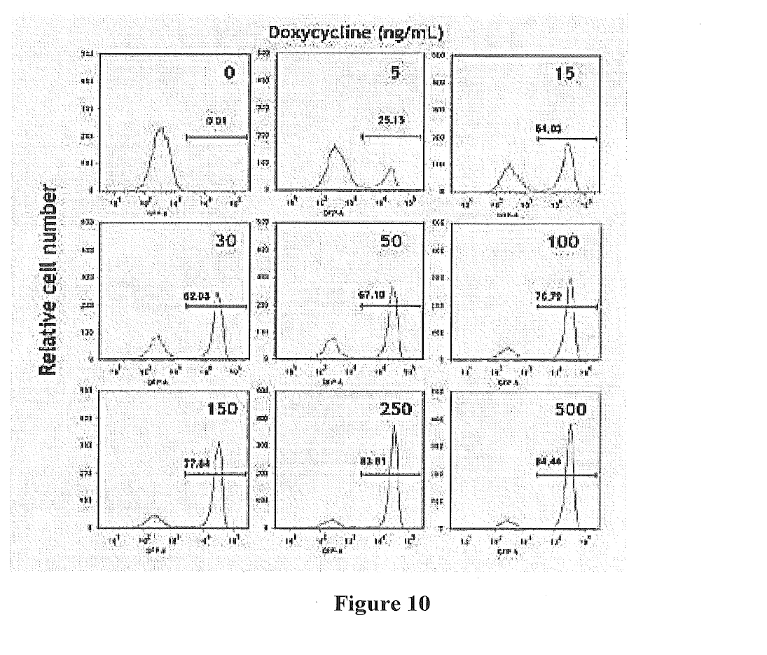

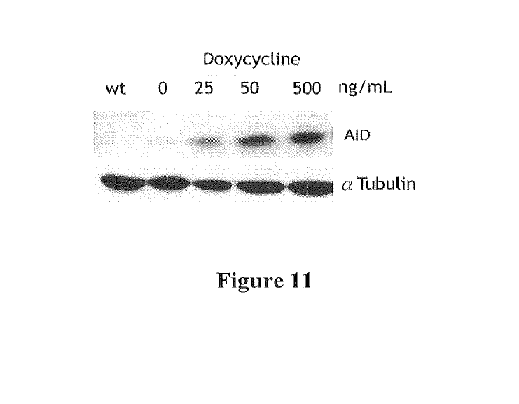

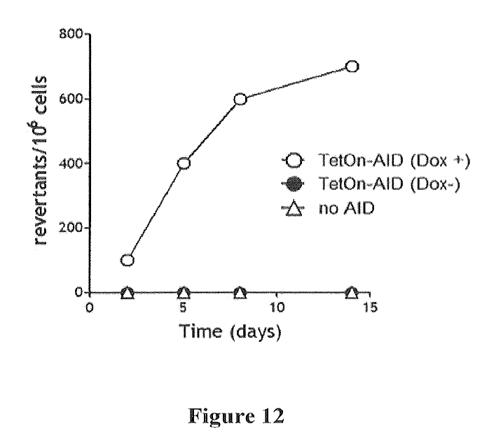

Controllable expression of AID in hybridomas was also achieved by using a tetracycline-inducible mAID expression cassette (TetOn-mAID) (FIG. 9). The autoregulatory TetOn-mAID is composed of the tetracycline response elements (TRE) promoter, a mAID gene, a HA epitope, a furin/2A peptide (F2A) bicistronic expression linker, an eGFP reporter gene, an IRES fragment and the rtTA-V14 transactivator. Addition of doxycycline (dox) activates rtTA-V14 transactivator to initiate TRE promoter driven gene expression encoding mAID, eGFP and rtTA-V14 transactivator. (Bottom) The structure of DsRed with a premature stop codon at nucleotide position 519 containing the hotspot motif of mAID. 3T3 finroblasts were infected with TetOn-mAID and selected in puromycin-supplemented medium. Addition of doxycycline to the stable 3T3 cells resulted in expression of the eGFP reporter in a dose-dependent fashion (FIG. 10), indicating successful regulation of AID expression in the 3T3 cells. FIG. 10 provides flow cytometry histograms of 3T3/TetOn-mAID fibroblasts induced with the indicated doxycycline concentration for 48 hours. Direct immunobloting of AID protein in the 3T3/TetOn-mAID hybridoma cells verified that AID could be controlled by the dose of doxycycline added to the culture medium (FIG. 11). The 3T3 cells were infected with lentiviral particles expressing Tet-on inducible AID. Hybridomas were exposed to the indicated concentrations of doxycycline to induce AID expression. Cell lysates were immunoblotted for AID or tubulin as a cell loading control. To verify mAID activity, we used sDsRed, possessing a premature stop codon (FIG. 9). 3T3 fibroblasts and 3T3/TetOn-mAID cells were stably transduced with sDsRed plasmid via retroviral infection. The somatic hypermutation activity of mAID was determined by quantifying the DsRed fluorescent revertants. DsRed fluorescence was only detected in the 3T3/TetOn-mAID transfectant with sDsRed DNA substrate after initiation of somatic hypermutation by addition of doxycycline (FIG. 12). In FIG. 12, 3T3 fibroblasts were transfected with tet-on inducible AID and a dsRED reporter containing a premature stop codon. The fraction of cells expressing red fluorescence (indicating mutation of the stop codon) versus time is shown. We conclude that mAID was active as shown by generation of mutations in the premature stop codon to rescue DsRed fluorescence.

### Somatic Hypermutation of a PEG-Specific Lineage

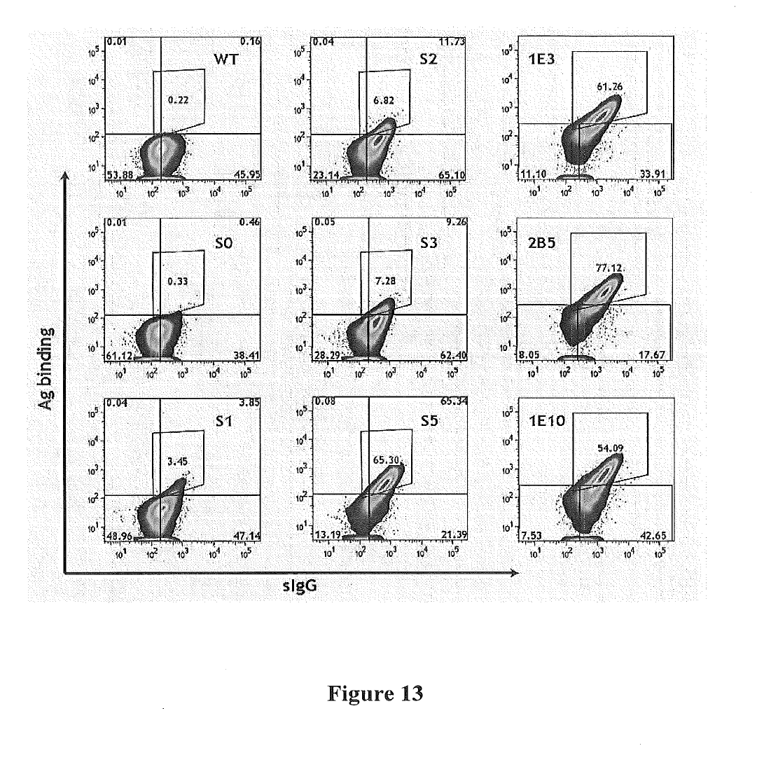

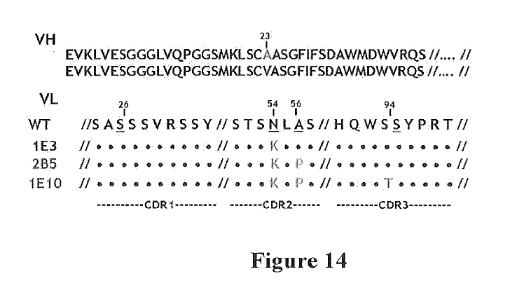

We generated stable 3.3 anti-PEG hybridoma cells (3.3/IoxP-AID cells) that express functional AID in a controllable manner by transducing a IoxP flanked constitutive expression cassette (pCMV-AID-IoxP) into 3.3 hybridoma cells. To test if mimicking the germinal center reaction could be used to isolate anti-PEG antibodies with altered binding activity, 3.3/IoxP-AID cells maintained on ice were stained with 100 pM of biotin-4arm-PEG_{10K} and Alexa Fluor 647-streptavidin. Cells (˜1% of the population) displaying the highest fluorescence were collected with a FACSAria cell sorter and expanded for 2 weeks. In subsequent rounds, the cells were stained with Alexa Fluor 647-PEG_{5K} or Alexa Fluor 647-BSA-PEG_{2K}. The expression levels of sIg on these hybridomas were also measured by co-staining cells with Alexa405-conjugated goat anti-mouse Ig antibody (x-axis). Cells displaying relative high antigen-binding capacity were collected during each round of sorting (S0 to S5). Selected hybridoma clones from the fifth round of sorting (1E3, 2B5 and 1E10) were also analyzed in the same way. We performed all PEG binding assays on ice to isolate anti-PEG antibody variants with enhanced binding at low temperatures. After five sequential rounds of sorting, isolated cell populations were stained with Alexa647-PEG_{5K} probes and analyzed on a flow cytometer. FIG. 13 shows that by the fifth round of sorting, the mean fluorescence intensity (MFI) of Alexa Fluor 647-PEG_{5K} binding to 3.3/IoxP-AID hybridoma cells was 233 as compared to a value of 23.2 for the parental 3.3 hybridoma cells, suggesting that these hybridoma cells expressed antibody variants that possessed enhanced PEG affinity at low temperature by the fifth round of in situ SHM Three hybridoma clones (1E3, 2B5 and 1E10) were isolated after the fifth round of sorting. Sequencing of the immunoglobulin V genes from these hybridomas showed that all three antibodies displayed a single amino acid change in the heavy chain variable framework region and multiple and variable mutations in CDR2 and CDR3 of the light chain variable region (FIG. 14). Antibody frameworks and CDRs were assigned according to Kabat numbering. The dashes indicate identical amino acid residues.

### A Shift in Thermoactivity of Anti-PEG Antibody Variants

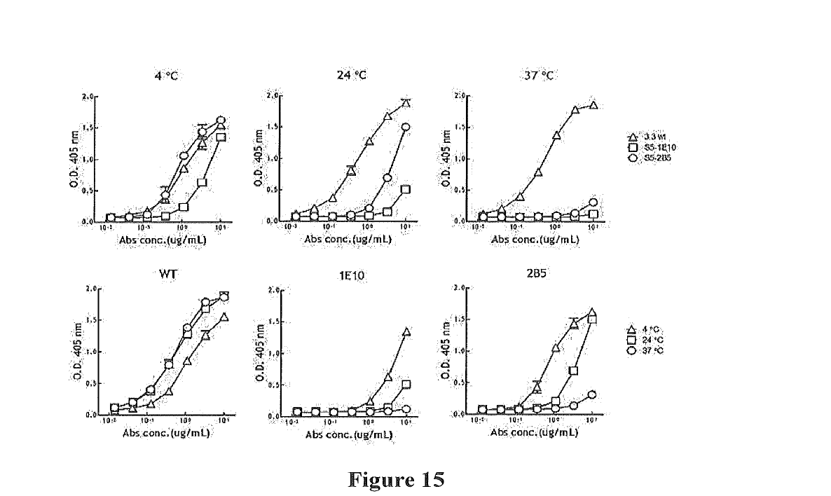

To further evaluate the binding activity of anti-PEG antibody variants (1E10 and 2B5) for the detection of PEG molecules compared to parental 3.3 antibodies, we analyzed the effect of temperature on the binding of these antibodies to PEG. Graded concentrations of 3.3, 1E10, or 2B5 antibodies were added to microplate wells coated with CH3-PEG5k-NH2 molecules at the indicated temperatures. After 1 h, the wells were washed and antibody binding was determined by adding HRP-conjugated donkey anti-mouse IgG Fc antibodies, followed by adding ABTS substrate. Both 1E10 and 2B5 antibodies bound to immobilized CH₃-PEG₅ₖ-NH₂ with full binding activity at 4° C. but displayed significantly decreased binding at higher temperatures (24° C. and 37° C.) (FIG. 15).

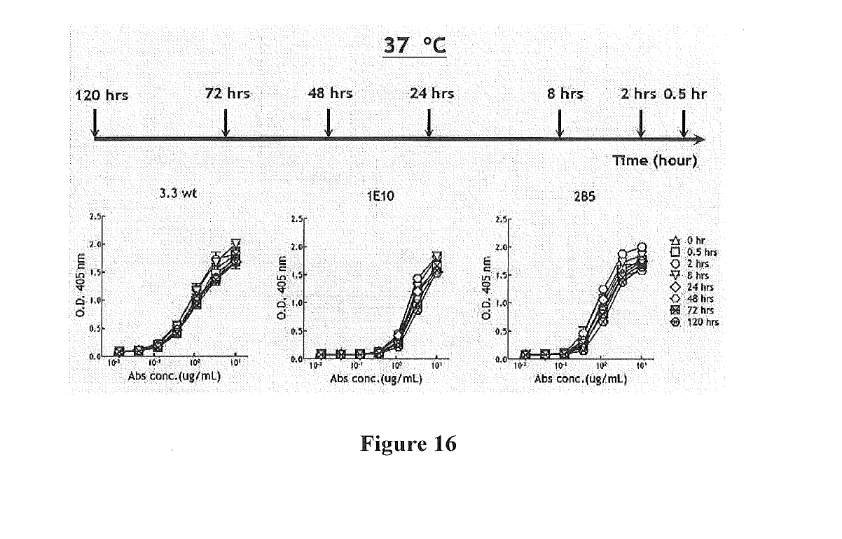

Binding of 1E10 and 2B5 antibodies to PEG therefore appears to be temperature-dependent. To confirm whether the thermoactivity shift resulted from antibody protein damage under higher temperatures, the stability of anti-PEG antibodies prepared from parental 3.3 or 3.3/S5 variants (1E10 and 2B5) was examined after incubation at 37° C. for up to 5 days. FIG. 16 shows that all the monoclonal antibodies retained full binding activity to absorbed PEG at 4° C. after incubation at 37° C. for 5 days. Antibodies were incubated at 37° C. for the indicated times. Aliquots of samples were examined for binding to absorbed CH3-PEG5k-NH2 molecules by ELISA. The mean absorbance values (405 nm) of triplicate determinations are shown. Bars, SD. Furthermore, determination of the melting temperatures of the antibodies by differential scanning calorimetry demonstrated that the parental 3.3 antibody unfolded at 70.8° C. whereas 1E3 and 2B5 were more thermostable with thermal transition temperatures of 73.6° C. and 74.2° C., respectively. These results show that these mAID induced mutations in the immunoglobulin V genes of these isolated monoclonal antibodies (1E10 and 2B5) contribute to the thermosensitive binding of 1E10 and 2B5 to PEG.

### Expression of Exogenous AID in FO Cells and Hybridomas

To further extend the utility of in situ somatic hypermutation to newly generated hybridomas, we asked whether it would be possible to transfect FO myeloma cells with an exogenous AID gene for use as a general fusion partner. FO myeloma cell lines produce no endogenous immunoglobulins and fuse effectively with B-lymphoblasts in the presence of polyethylene glycol. We expect that hybridomas generated by fusing FO-AID cells with splenocytes can undergo somatic hypermutation and high affinity clones can be easily sorted after multiple rounds of selection and enrichment using flow cytometry.

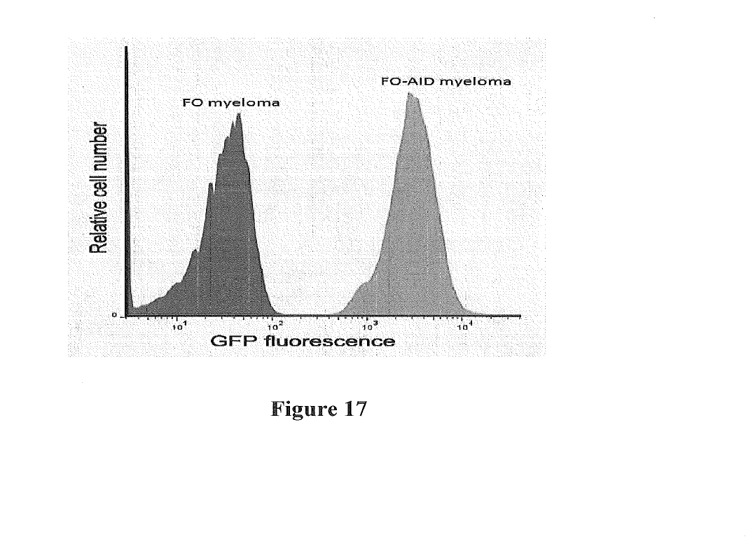

We transduced FO myeloma cells with recombinant lentivirus particles containing the IoxP-CMV-mAID-HA-F2A-eGFP-IoxP expression cassette to allow constitutive expression of murine AID in these cells. eGFP is also expressed as a reporter via a furin/2A peptide (F2A) bicistronic sequence downstream of the AID gene. Constitutive expression of mAID was detected in the stable FO myeloma cell transfectants (FO/AID cells) (FIG. 17). The fluorescence of the GFP reporter in FO (left filled curve) and FO/AID myeloma cells (right filled curve) is shown.

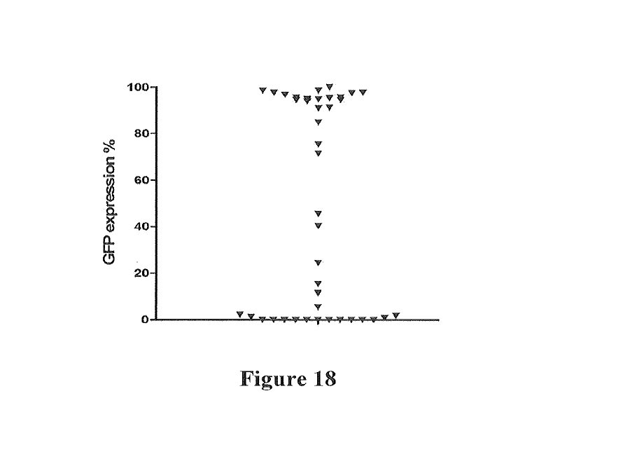

To test if AID would also be expressed in hybridomas generated by fusing FO/AID myeloma cells with splenocytes, BALB/c mice were immunized intraperitoneally with 50 μg recombinant human beta-glucuronidase in Freund's Complete Adjuvant. Three weeks later, immunizations were repeated using 40 μg recombinant human beta-glucuronidase in Freund's incomplete adjuvant. Three days prior to fusion, 30 μg recombinant human beta-glucuronidase in PBS was given intraperitoneally as the final boost. On the day of fusion, cells were prepared from immunized mice and fused with FO/AID cells. Fusion was performed using polyethylene glycol. Following fusion, cells were plated in 96-well plates and maintained in Dulbecco's modified Eagle's medium (Sigma, St Louis, Mo., USA) with 15% bovine calf serum (HyClone, Logan, Utah) and 1× hypoxanthine-aminopterin-thymidine (HAT, Gibco, Brooklyn, N.Y.). Supernatants of growing hybridomas were screened by ELISA in 96 well plates coated with 0.5 μg human beta-glucuronidase per well for 1 h, then wells were washed and specific antibody binding was determined by adding HRP-conjugated donkey anti-mouse IgG Fc antibodies, followed by adding ABTS substrate. About 63% of the hybridomas generated by fusion of FO/AID cells with splenocytes from mice immunized with human beta-glucuronidase (26/41) show positive GFP expression (FIG. 18), indicating that the AID gene is sequestered at the expected frequency for unbiased gene segregation. The relative expression of GFP in each hybridoma among 41 hybridoma clones is given in the figure.

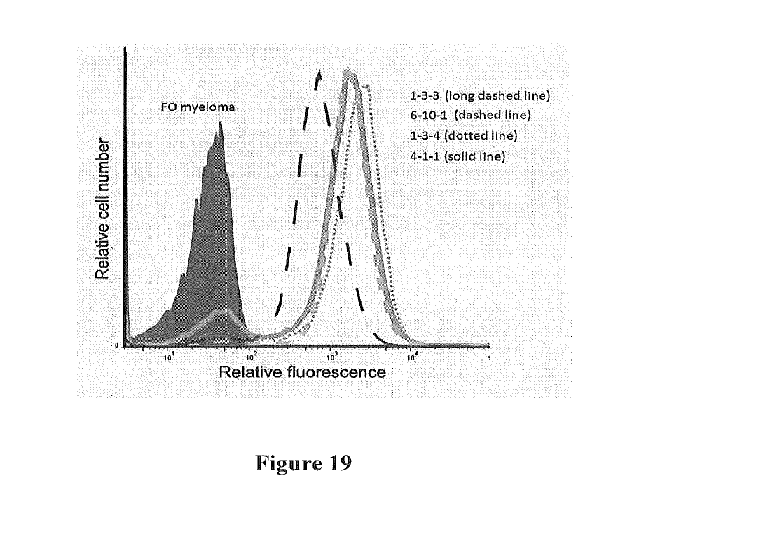

Individual hybridomas that secreted monoclonal antibodies with specificity to human beta-glucuronidase were selected and maintained in puromycin (5 μg/mL). Four clones were chosen (1-3-4, 1-3-3, 6-10-1, and 4-1-1) to be cultured in large scale for further analysis and evolution. The relative expression level of mAID in hybridomas was measured on a FACScalibur flow cytometer (Becton Dickinson, Mountain View, Calif., USA) by detecting the expression of the eGFP reporter. FIG. 19 shows the expression of GFP reporter in FO (left filled curve) and 1-3-3 (solid line), 6-10-1 (dashed line), 4-1-1 (complex line), 1-3-4 (dotted line) hybridoma cells. As expected, all hybridomas that were selected in puromycin expressed GFP (FIG. 19).

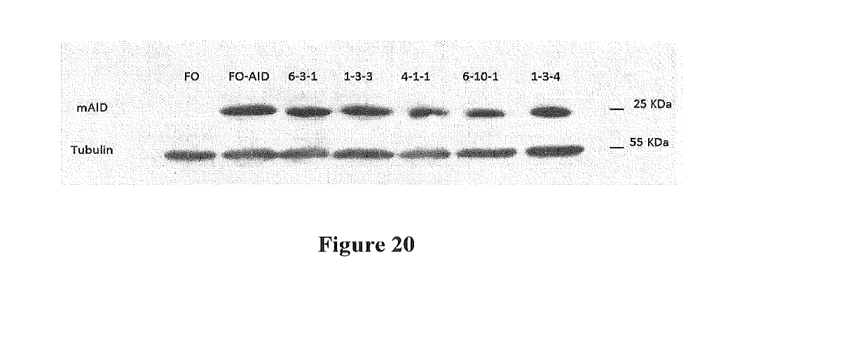

To directly measure the expression of mAID protein, Western blotting was performed for five hybridomas (6-3-1, 1-3-4, 1-3-3, 6-10-1, and 4-1-1), in addition to the parental FO-AID myeloma cells. Cell lysates prepared from FO, FO-AID and five anti-beta-glucuronidase hybridomas (prepared from FO/AID as a fusion partner) were immunoblotted for the HA epitope tag on AID or tubulin as a loading control. All five selected hybridomas as well as FO-AID myeloma cells showed a specific band corresponding to mAID (FIG. 20). By contrast, parental FO myeloma cells did not express mAID. This result confirms that hybridomas generated by fusion of mouse splenocytes with FO-AID myeloma cells can express the AID protein.

Expression of Antigen-Specific Antibodies from the Selected Hybridomas

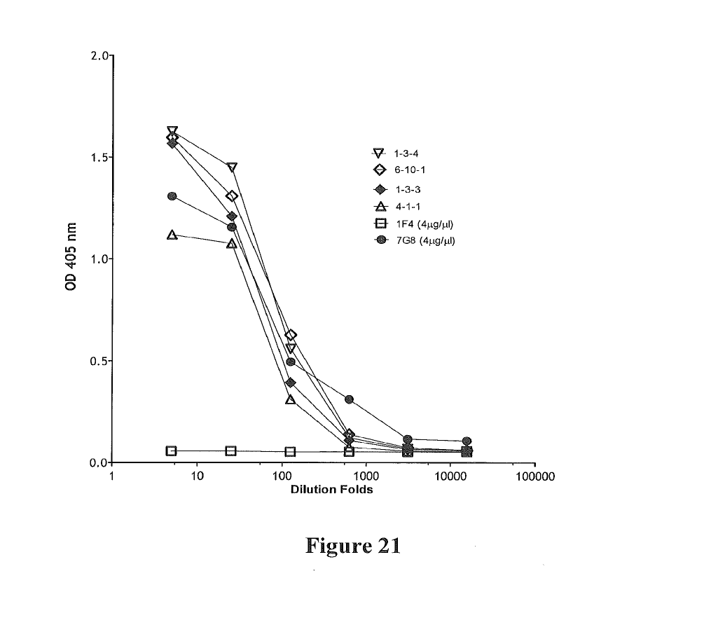

ELISA was used to evaluate the binding activity of antibodies secreted from the selected hybridomas (1-3-4, 1-3-3, 6-10-1, and 4-1-1). Cell culture medium from four hybridoma clones (1-3-4, 1-3-3, 6-10-1, and 4-1-1) were analyzed by direct ELISA. 7G8 anti-human beta-glucuronidase and 1F4 anti-human CD13 monoclonal antibodies were used as positive and negative controls respectively. Samples or controls were serially diluted, and then equal volumes were added to microplate wells coated human beta-glucuronidase. Antibody binding was determined by adding HRP-conjugated donkey anti-mouse IgG Fc antibodies, followed by adding ABTS substrate. All four hybridomas expressed antibodies that bound human beta-glucuronidase (FIG. 21). As expected, the previously established 7G8 anti-beta-glucuronidase antibody bound to wells coated with beta-glucuronidase while the 1F4 anti-CD13 antibody did not bind.

### Affinity Maturation of Anti-Human Beta-Glucuronidase Hybridomas

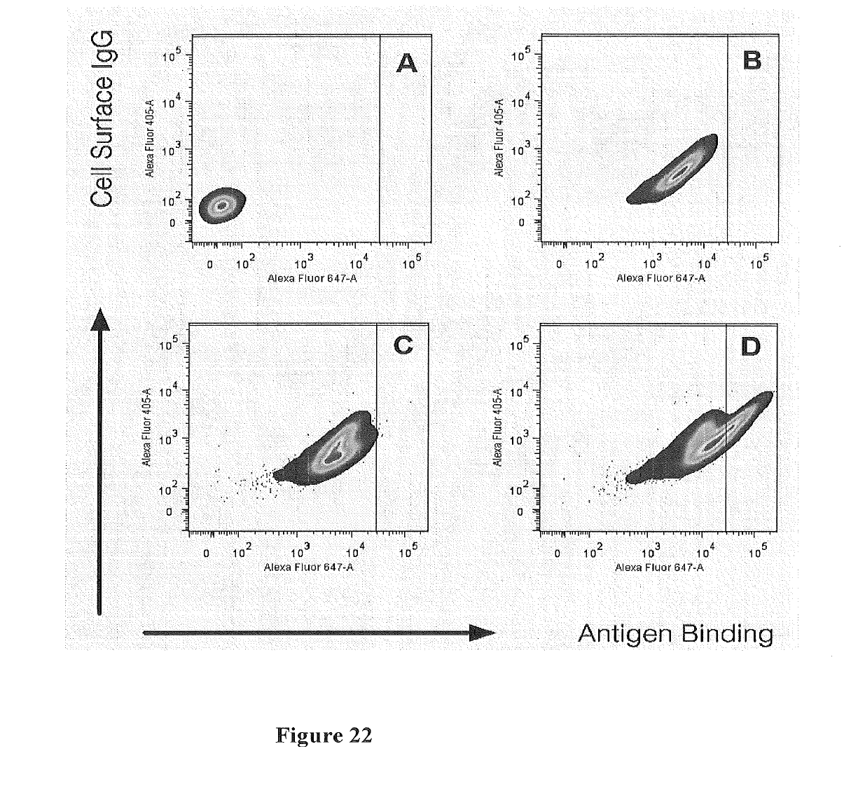

The 1-3-4 hybridoma was selected to examine if antigen-positive cells could be isolated by fluorescence-activate cell sorting. Cells were stained with a limiting concentration of beta-glucuronidase-Alexa647 starting from 1 nM and decreasing to the concentration by half sequentially, and then sorted on FACS Aria cell sorter. After five sequential rounds of sorting, unsorted parental 1-3-4 cells and sorted cells (1-3-4-S5 cells) were stained with 200 nM of beta-glucuronidase-Alexa647. The expression of surface immunoglobulin on the hybridomas was also determined by co-staining with 200 nM Alexa405-conjugated goat anti-mouse Ig antibody. Cells were then analyzed by flow cytometer. In FIGS. 22A through 22D, the panels are directed toward the following: FO-AID cells (A), 5G11 anti-human beta-glucuronidase hybridoma (B), unsorted 1-3-4 parental hybridoma (C), and 1-3-4 after 5 sorting rounds (1-3-4-S5) (D). FO-AID cells displayed neither antigen binding nor surface immunoglobulin expression (FIG. 22A), while the positive control 5G11 hybridoma cells had both antigen binding and surface immunoglobulin expression (FIG. 22B). 1-3-4 hybridoma cells that were selected in five rounds of sorting (1-3-4-S5 cells) exhibited a brighter signal for antigen binding (FIG. 22D) as compared to the parental 1-3-4 hybridoma cells (FIG. 22C), suggesting that this strategy can be employed to isolate monoclonal antibodies with enhanced affinity.

### Increasing the Affinity of an IgM Anti-PEG Antibody by Mimicking the Germinal Center Reaction

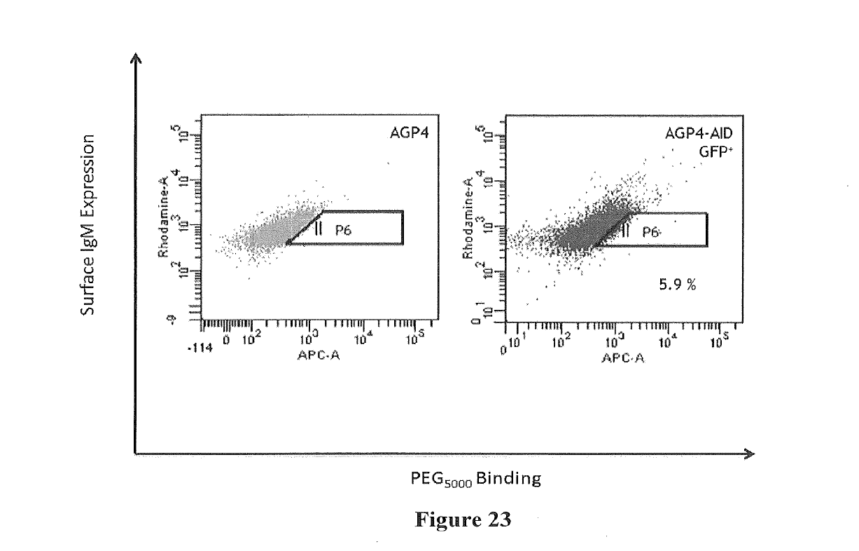

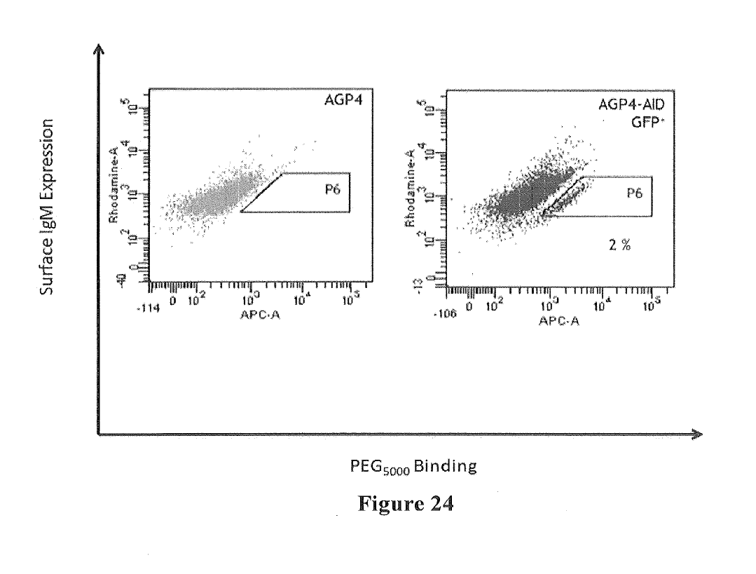

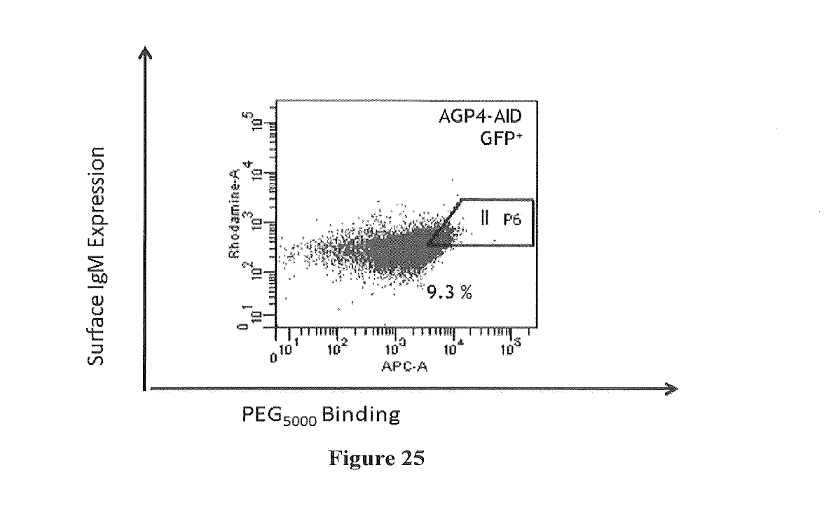

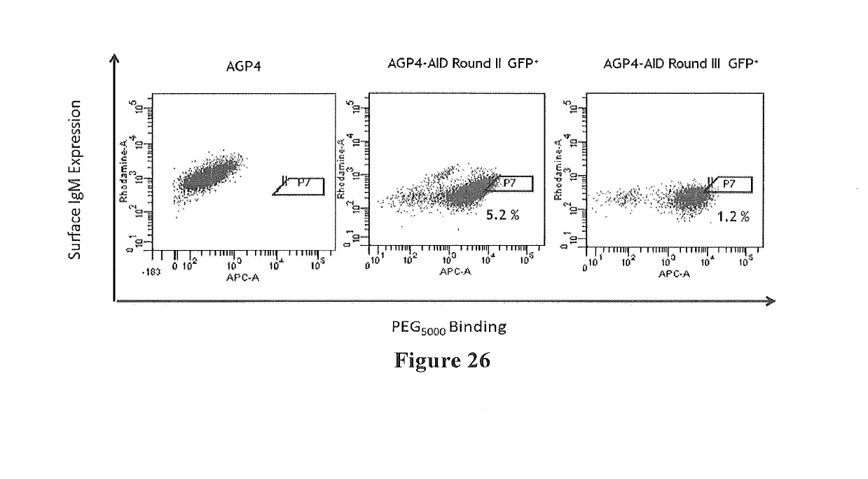

We performed experiments to directly test if the affinity of an IgM anti-PEG antibody could be increased by mimicking the germinal center reaction. AGP4 anti-PEG hybridoma cells that stably express the AID gene (by lentiviral transduction of pCMV-AID-IoxP) were sorted on a fluorescence-activated cell sorted for cells that bound to limiting concentration of PEG-biotin. The hybridoma cells were cultured for two weeks to allow somatic hypermutation of AGP4 antibody genes. The hybridoma cells were then stained with a mixture of 10 nM biotin-PEG5000 and 20 nM streptavidin-APC (fluorescence-labeled streptavidin) to select high affinity AGP4 antibodies on the surface of the hybridoma cells as well as with rhodamine-labeled anti-mouse mu-chain antibody to measure the amount of AGP4 IgM antibodies on the surface of the hybridoma cells. Hybridoma cells that displayed high APC fluorescence, corresponding to binding of more PEG, were sorted into a test tube (FIG. 23). FIG. 23 shows staining of parental AGP4 (left) or AGP4-AID (right) hybridoma cells for binding to 10 nM biotin-PEG₅₀₀₀, 20 nM Streptavidin-APC+rhodamine-anti-Mo IgM μ-chain. AGP4-AID cells in the gated area (P6) were collected and cultured to expand the cell number and for continued somatic hypermutation of the AGP4 antibody. The collected hybridoma cells were then cultured and expanded for two weeks to allow additional somatic hypermutation of the AGP4 genes. The hybridoma cells were again stained as before and high PEG binders were again collected by fluorescence-activated cell sorting (FIG. 24). FIG. 24 shows staining of parental AGP4 (left) or AGP4-AID (right) hybridoma cells for binding to 10 nM biotin-PEG₅₀₀₀, 20 nM streptavidin-APC+rhodamine-anti-Mo IgM μ-chain. AGP4-AID cells in the gated area (P6) were collected and cultured for further somatic hypermutation of the AGP4 antibody. After 2 weeks the cells were stained, but with less antigen (3.3 nM PEG-biotin) to select for high PEG binders (FIG. 25). FIG. 25 shows staining of AGP4-AID hybridoma cells (collected after two rounds of screening) for binding to 3.3 nM biotin-PEG₅₀₀₀, 6.6 nM Streptavidin-APC+rhodamine-anti-Mo IgM μ-chain. AGP4-AID cells in the gated area (P6) were collected and expanded for continued somatic hypermutation of the AGP4 antibody. The highest binders were collected and then analyzed by FACs to determine if antibody affinity had increased. This was accomplished by staining, under conditions of limiting fluorescence-labeled antigen, the original AGP4 hybridoma cells as well as the AGP4-AID hybridoma cells collected after round II or round III of sorting. FIG. 26 shows that the cells collected after round II or round III bound much higher levels of fluorescence-labeled PEG as compared to the original AGP4 hybridoma cells. In the figure, parental AGP4 (left) or AGP4-AID hybridoma cells after two rounds of selection (middle panel) or three rounds of selection (right panel) were stained with fluorescence-labeled antigen under identical conditions (3.3 nM biotin-PEG₅₀₀₀, 6.6 nM streptavidin-APC+rhodamine-anti-mouse IgM μ-chain). Notice that IgM expression on the hybridoma cells decreased, suggesting class switch to IgG.

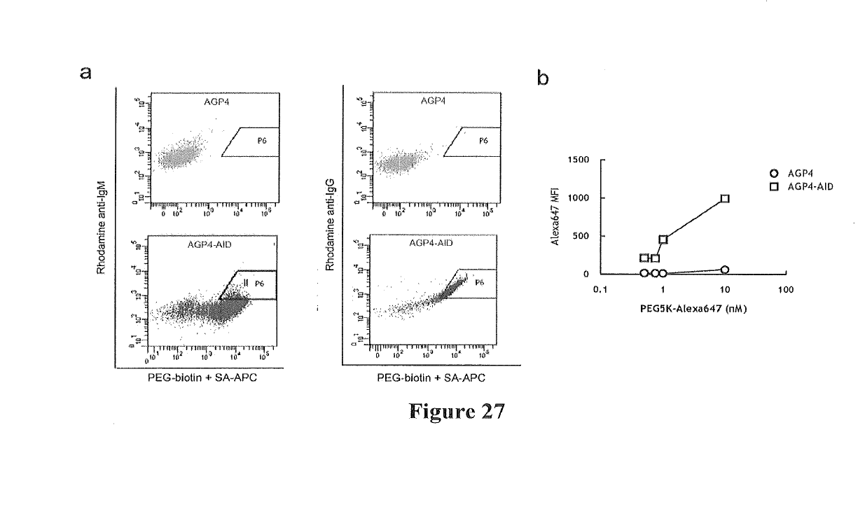

We noticed that the expression of IgM on the surface of the AGP4 hybridoma cells appeared to decrease with each round of selection. This suggested that the AGP4 antibody may have undergone class switch recombination. FIG. 27 shows the result of a, staining of parental AGP4 or AGP4-AID hybridoma cells (after 3 rounds of sorting) for IgM (left) or IgG (right) as well as for binding to PEG (x-axis), and b, FACs analysis (MFI) of PEG-Alexa 647 binding to AGP4 or AGP4-AID cells after 3 rounds of sorting. Parental AGP4 hybridoma cells or AGP4-AID hybridoma cells after three rounds of selection, were stained for PEG binding as well as for the presence of IgM or IgG antibody on the surface of the hybridoma cells. A clear population of hybridoma cells that expressed IgG that bound PEG with high affinity was clearly evident in the AGP4-AID cells after three rounds of sorting (FIG. 27a, bottom right panel). We also stained the AGP4-AID hybridoma cells collected after three rounds of sorting, but with an alternative PEG molecule to ensure that selected AGP4 antibodies indeed bound PEG with high affinity. As shown in FIG. 27b, AGP4-AID cells after three rounds of sorting could bind PEG-Alexa647 much better than the parental AGP4 hybridoma cells. Importantly, hybridoma cells still expressed sufficient antibody on their surface for FACs screening after class switch from IgM to IgG. We conclude from these results that we can mimic the germinal center reaction to increase the affinity of an IgM antibody as well as perform class switch recombination to generate an IgG antibody with the same antigen-binding specificity.

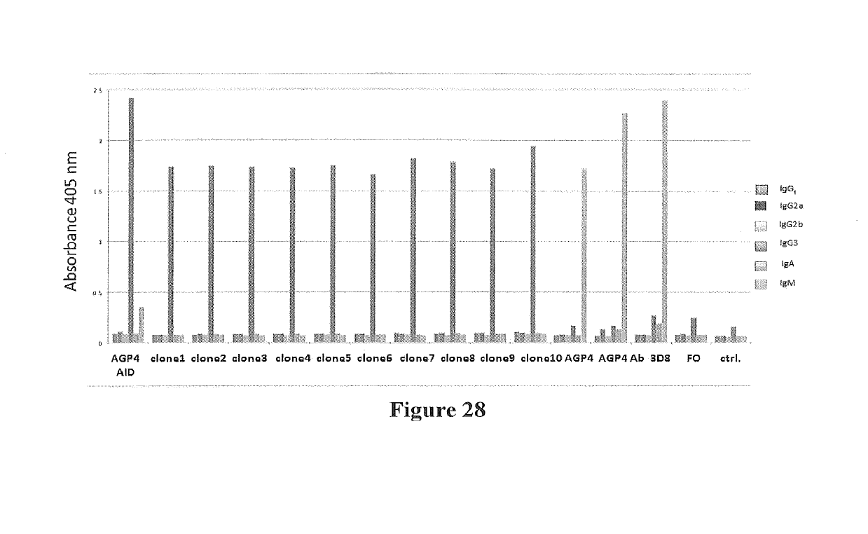

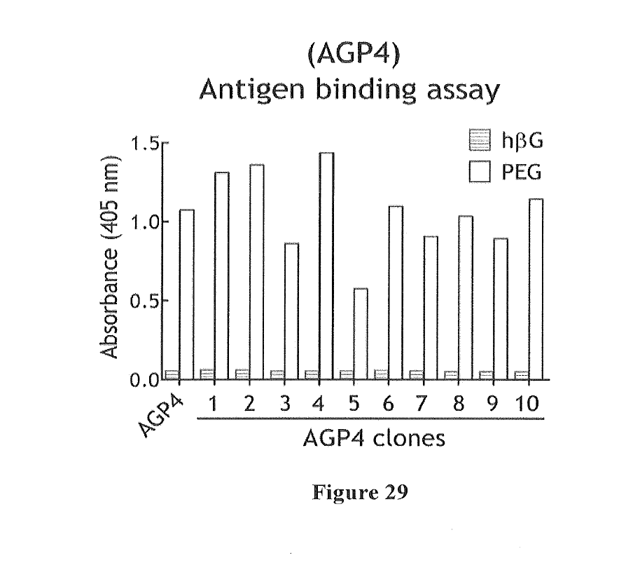

We wished to further verify that class switch recombination from IgM to IgG had occurred in AGP4-AID cells. We therefore sorted by FACs individual AGP4-AID cells (after three rounds of sorting) based on ability to bind PEG and express IgG on their surface. Individual cells were sorted directly into 96-well culture plates and allowed to grow for about 10 days. The secreted AGP4 antibody in the wells was then assayed using an isotype ELISA to determine the heavy chain class of the antibodies. All ten clones assayed were found to be IgG₃ (FIG. 28), verifying that AGP4 IgM did undergo class switch to IgG. FIG. 28 shows that after three rounds of somatic hypermutation and sorting, ten hybridoma clones that appeared to display surface IgG were collected as single cells and expanded. The class of AGP4 antibody in the culture supernatants of the hybridoma cells was assayed by ELISA. All ten AGP4 hybridoma clones secreted IgG3 antibodies, demonstrating successful class switch of AGP4 from IgM to IgG. All AGP4 IgG antibodies retained the ability to bind PEG (FIG. 29), verifying that they were derived from AGP4. In FIG. 29, class switched AGP4 IgG was assayed for binding to PEG coated in 96-well plates by ELISA. All AGP4 IgG antibodies retained antigen (PEG) binding activity and none of the antibodies bound to control antigen (beta-glucuronidase), indicating retention of specificity. We conclude that mimicking the germinal center reaction in a test tube is a convenient method to both increase the affinity of a monoclonal antibody as well as to switch the class of the antibody.

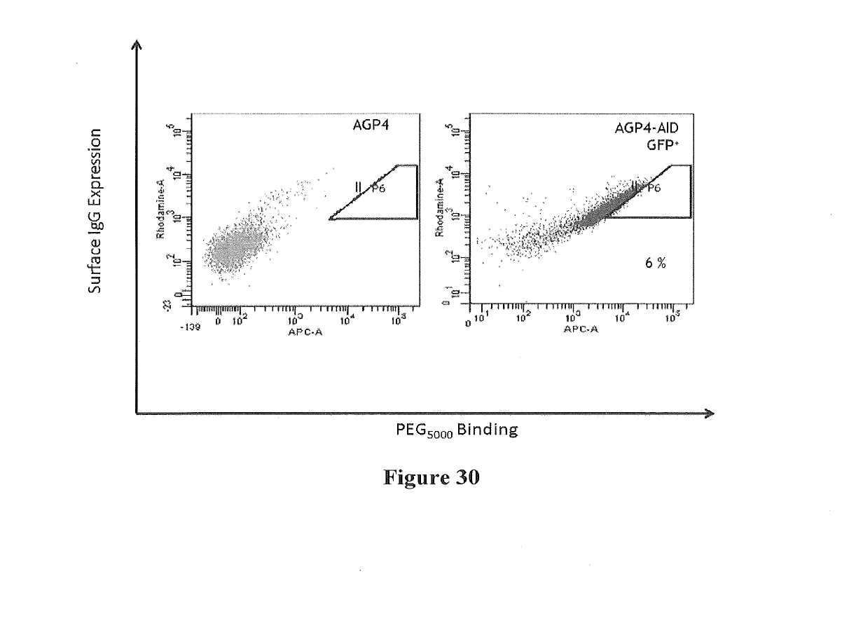

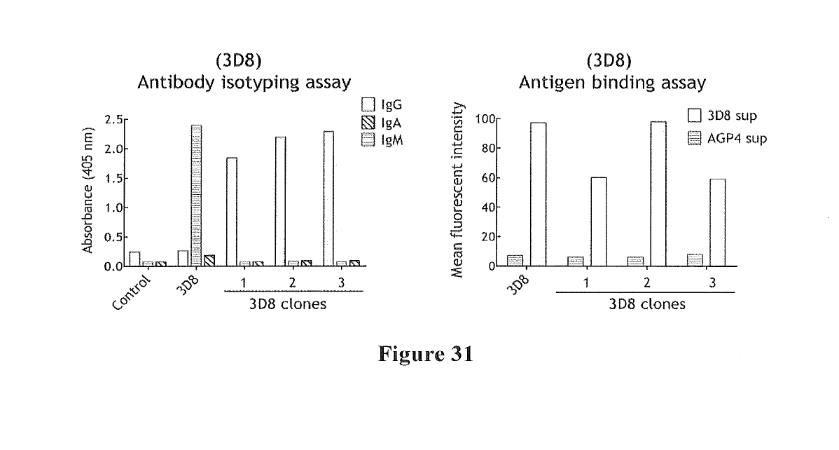

AGP4 IgG was further affinity matured by staining the cells with 1 nM biotin-PEG and 2 nM streptavidin-APC in a mixture with rhodamine-labeled anti-mouse IgG (FIG. 30). FIG. 30 shows staining of parental AGP4 (left) or AGP4-AID (right) hybridoma cells for binding to 1 nM biotin-PEG₅₀₀₀, 2 nM Streptavidin-APC rhodamine-anti-Mo IgG. AGP4-AID cells that expressed surface IgG and bound high levels of fluorescence-labeled PEG in the gated area (P6) were collected and expanded for continued somatic hypermutation of the AGP4 IgG antibody. Thus affinity maturation of IgG is also feasible. We also tested if another IgM antibody could be class switched to IgG. We stably expressed the AID gene in 3D8 hybridoma cells, which secrete an IgM antibody that can bind a surface antigen expressed on B16 melanoma cells. After 2 weeks, 3D8 hybridoma cells were stained with fluorescence-labeled anti-mouse IgG antibody and positive cells were individually sorted by FACs. After the cells expanded, assay of the antibody in the culture medium revealed that all three clones secreted IgG (FIG. 31, left panel). The IgG antibodies retained the ability to bind to B16 melanoma cells (FIG. 31, right panel). In FIG. 31, 3D8 hybridoma cells were infected with lentivirus expressing the AID gene. After culture for several weeks, hybridoma cells that expressed membrane IgG, as determined by FACs, were sorted as single cells into 96-well plates. The class of antibody in the culture medium from three individual clones was determined by ELISA to be IgG (left panel). Class switched 3D8 IgG from the three hybridoma clones was assayed for binding to B16 melanoma cells by FACs (right panel). All 3D8 IgG antibodies retained antigen binding activity as shown by binding to the melanoma cells. As a binding control, AGP4 antibodies did not bind to the melanoma cells. We conclude that class switch from IgM to IgG can be rapidly and easily performed by our methodology.

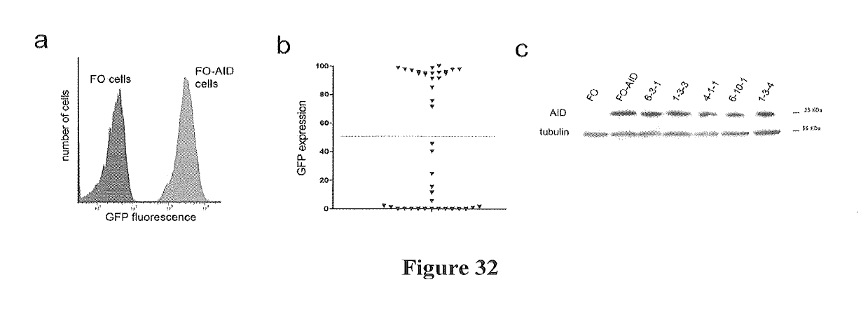

To make new hybridoma cells with the built in capacity to perform in situ somatic hypermutation and class switch recombination, we prepared FO myeloma cells that stably express AID in a controllable fashion (FIG. 32a). FO myeloma cells are often used as a fusion partner to make hybridoma cells. Fusion of the FO-AID cells with splenocytes from a mouse immunized with a protein antigen revealed that about 50% of the resulting hybridoma cells expressed GFP, which is indicative of successful expression of AID in these hybridoma cells (FIG. 32b). Hybridoma cells that express AID can be selected by addition of purimycin to the culture medium. We performed limiting dilution cloning of several hybridoma cells and then performed immunoblotting to prove that these newly formed hybridoma cells did express AID protein (FIG. 32c). FIG. 32 shows a, Staining of parental FO or FO-AID myeloma cells for GFP, which represents AID expression, b, FACs analysis of GFP expression in hybridoma cells formed by fusion of mouse splenocytes with FO-AID cells, and c, the expression of AID in FO, FO-AID and individual hybridoma clones as determined by immunoblotting cell lysates with an antibody against AID or an antibody against tubulin as a cell loading control. These results show that we can generate new hybridoma cells with the built in capacity to perform affinity maturation and class switch of specific monoclonal antibodies.

### Human Antibodies by Mimicking the Germinal Center Reaction

Mimicking the germinal center reaction in hybridoma cells can produce high affinity greatly human monoclonal antibodies, which are important for the treatment of many diseases. Current methods to generate human monoclonal antibodies include using phage display libraries or generating antibody-producing hybridoma cells from patient B cells or from immunized transgenic antibody mice. Phage display is a powerful method to generate human antibodies but suffers from drawbacks, including the need to construct large cDNA libraries, the requirement for additional engineering, immunogenicity and their tendency towards instability and aggregation, which can cause trouble with antibody formulation and storage.^{26,54,55} Human antibodies can also be directly generated by immortalization or fusion of B cells isolated from human subjects. Indeed, great progress has been made to improve the efficiency of EBV immortalization of human B cells after it was discovered that addition of the TLR9 agonist CpG can increases the efficiency of B cell immortalization from 1-2% to 30-100%.^{56,57} Although immortalization is effective, antibody production levels tend to be low. Better cloning efficiency, stability and antibody production levels can be achieved by fusing EBV-immortalized B cells with mouse-human heteromyeloma cells by standard hybridoma technology.⁵⁸ However, current human hybridoma technology is mostly limited to making antibodies against vaccinated antigens where the frequency of antigen-positive B cells is sufficient to isolate specific antibody-producing hybridoma cells.⁵⁸⁻⁶⁰

Human immune mice represent a promising avenue to human monoclonal antibody production that is widely assessable to both biotechnology companies and scientific laboratories. Human immune mice can be generated by injecting CD34⁺ hematopoietic stem cells (isolated from cord serum) into NOD/SCID/IL-2Rγ^{null} (NSG) mice. Intrahepatic injection of CD34⁺ human cord cells into conditioned newborn NSG mice can reconstitute up to 40% human CD45⁺ cells in peripheral blood, 60% in bone marrow, 69% in the spleen and up to 95% in the liver after 20 weeks.^{61,62} Mature B cells were prominent in the liver and spleen and human monocytes, macrophages, dendritic cells and NK cells were detected in the liver, spleen and bone marrow.⁶¹ Human immune mice also develop a highly diverse T cell repertoire and produce robust T cells antigen-specific CD4⁺ and CD8⁺ T cell responses.^{62,63} These mice are becoming increasingly important in the study of hematopoiesis, infectious diseases, autoimmunity and cancer immunology.⁶⁴ Importantly, humanized mice can form humoral immune responses against administered antigens including human proteins,⁶⁵⁻⁶⁷ thereby allowing the isolation of human monoclonal antibodies.⁶⁸

A major roadblock in the widespread use of human immune mice for the generation of human monoclonal antibodies is a propensity for a predominantly low affinity IgM antibody response from these mice.^{64,68,69} B1-like B cells, which are responsible for secretion of “natural IgM antibodies”,⁷⁰ are believed to preferentially develop in the hematopoietic environment in reconstituted human immune mice.⁷¹ Several methods have been proposed to increase the IgG response in human immune mice, including administration of B cell cytokines,⁷² engrafting MHC class II matched human stem cells to MHC transgenic NSG mice,⁷³ and T cell adoptive transfer.⁷⁴ However, IgG responses are still suboptimal even with these manipulations.⁶⁴

We can overcome current limitations in producing human monoclonal antibodies in human immune mice. Fusion of the AID-expressing myeloma cells with EBV-immortalized splenic B cells from previously immunized human immune mice will thus produce human antibody hybridoma cells with a built in capability to turn on AID expression for “on demand” induction of somatic hypermutation of antibody variable region genes as well as for class switch recombination. As shown in our results, expression of AID in hybridoma cells can mimic the germinal center reaction to facilitate antibody affinity maturation and heavy chain class switch. This will help solve the major bottleneck of producing human monoclonal antibodies in human immune mice.

### Materials and Methods

### Cell Lines and Reagents

BALB/3T3 mouse fibroblasts (CCL-163), CC49 (IgG₁ mAb against TAG-72, HB-9459), L6 (IgG₂ₐ mAb against human L6 antigen, HB-8677), BC3 (IgG_{2b} mAb against human CD3 epsilon chain, HB-10166), and PEG-1-6 (IgG₃ mAb against influenza virus, CCL-189) hybridoma cells were purchased from American Type Culture Collection (ATCC, Manassas, Va.). Hybridoma cell lines AGP4 (IgM mAb against polyethylene glycol), 3.3 and 6-3 (IgG₁ mAbs against polyethylene glycol), 7G8 (IgG₁ mAb against human beta-glucuronidase), and 3D8 (IgM mAb against B16F0 melanoma) were developed in our lab and have been described^{16, 39}. Human 293FT cells were kindly provided by Dr. Ming-Zong Lai (Institute of Molecular Biology, Academia Sinica, Taiwan). All cells were cultured in Dulbecco's modified Eagle's medium supplemented with 2.98 g/L HEPES, 2 g/L NaHCO₃, 10% fetal calf serum (HyClone, Logan, Utah), 100 U/mL penicillin and 100 μg/mL streptomycin at 37° C. in a humidified atmosphere of 5% CO₂ in air. Methoxy-PEG₇₅₀-N H₂, methoxy-PEG_{1K}-N H₂, methoxy-PEG_{2K}-N H₂, methoxy-PEG_{3K}-NH₂ hydroxy-PEG_{5K}-NH₂, methoxy-PEG_{10K}-NH₂, methoxy-PEG_{20K}-N H₂ (750, 1000, 2000, 3000, 5000, 10000 and 20000 Da, respectively), 4-arm poly(ethylene oxide)_{10K}-NH₂, and 18-crown-6 were purchased from Sigma-Aldrich.

### DNA Plasmid Construction

pAS4w.1.Ppuro, pAS3w.Ppuro, pAS3w.Pneo, pLKO.AS2.eGFP, pMD.G (VSV-G envelope plasmid) and pCMVΔR8.91 (packaging plasmid) vectors were obtained from the National RNAi Core Facility (Institute of Molecular Biology/Genomic Research Center, Academia Sinica, Taiwan). To generate a stoppable AID expression system, we designed a IoxP flanked IoxP-CMV-AID-HA-F2A-eGFP-IoxP expression cassette (pCMV-AID-IoxP). A HA-tagged murine activation-induced deaminase (AID-HA) DNA fragment was cloned from splenocytes isolated from BALB/c mice by RT-PCR. To monitor the expression of AID-HA, a furin-2A (F2A) based bicistronic expression strategy was used to link an enhanced green fluorescence protein (eGFP gene) downstream of the mAID-HA gene. The HA-F2A-eGFP fragment containing part of the HA tag and eGFP gene was amplified from the pLNCX-anti-PEG-eB7 vector⁴⁰. The eGFP fragment was cloned by PCR from the pLKO.AS2.eGFP. The AID-HA-F2A-eGFP gene was then created by assembly PCR from AID-HA and F2A-eGFP fragments and inserted into the pAS3w.Ppuro plasmid. To introduce IoxP sites, annealed oligonucleotides were inserted into a Spe I site upstream of the CMV promoter and in a Pme I site downstream of eGFP, respectively. We also constructed an inducible AID expression vector. rtTA-M2 was amplified from pRetroX-Tet-On Advanced (Clontech, Mountain View, Calif.) by PCR and then mutated using multisite-directed mutagenesis⁴¹ to obtain the rtTA-V14 gene.⁴² An IRES-rtTA-V14 fragment was generated by assembly PCR. A Nhe Pme I digested mAID-HA-F2A-eGFP fragment and the IRES-rtTA-V14 fragment were inserted into pAS4w.1.Ppuro to create the pAS4w.1.Ppuro-AID-F2A-eGFP-IRES-rtTA-V14 plasmid, denoted as pTetOn-AID. A DsRed2 DNA fragment amplified from pDsRed2 (Clontech Laboratories, Inc., Mountain View, Calif., USA) was inserted into pAS3w.Pneo to generate pAS3w.Pneo-DsRed. An amber stop codon was introduced into pAS3w.Pneo-DsRed at nucleotide position 519 by site directed mutagenesis using a QuikChange™ Site-Directed Mutagenesis Kit (Stratagene, Santa Clara, Calif.) to generate p-sDsRed2.

### Biotinylation of PEG and Beta-Glucuronidase

4arm-PEG_{10K}-NH₂, methoxy-PEG_{5K}-NH₂ and methoxy-PEG_{2K}-NH₂ molecules (Laysan Bio, Arab, Ala.) dissolved in DMSO at 2 mg/mL were mixed with a 6-fold (for 4arm-PEG_{10K}-NH₂) or 2-fold (for methoxy-PEG_{5K}-NH₂ and methoxy-PEG_{2K}-NH₂) molar excess of EZ-link NHS-LC-Biotin (Pierce, Rockford, Ill.) or Alexa Fluor® 647 succinimidyl esters (Invitrogen, Grand Island, N.Y.) (in DMSO) for 2 h at room temperature to produce biotinylated 4arm-PEG_{10K} or Alexa Fluor 647 conjugated methoxy-PEG_{5K} and methoxy-PEG_{2K}, respectively. These compounds were diluted in a 5-fold volume of ddH₂O and dialyzed against ddH₂O to remove free EZ-link NHS-LC-Biotin or Alexa Fluor 647. Likewise, human beta-glucuronidase was dissolved in PBS (pH 8.0) at 2 mg/mL and then mixed with a 20-fold molar excess of EZ-link NHS-LC-Biotin for 2 h at room temperature to produce biotinylated beta-glucuronidase. One-tenth volume of 1 M glycine solution was added to stop the reaction. Biotinylated beta-glucuronidase was dialyzed against PBS to remove free EZ-link NHS-LC-biotin, sterile filtered and stored at −80° C.

### Analysis of Membrane-Bound Immunoglobulin on Hybridoma Cells

Surface expression of mouse immunoglobulin (B cell receptors, BCR) on hybridoma cells was measured by staining cells with 2 μg/mL of goat anti-mouse Ig (ICN Pharmaceuticals, Inc, CA, USA) or goat anti-E. coli antibody (Abcam, Mass., USA) as a negative control in PBS containing 0.05% BSA at 4° C. for 1 hour. The cells were washed three times with cold PBS and stained with 2 μg/mL FITC-conjugated rabbit F(ab)′₂ anti-goat antibody (ICN Pharmaceuticals, Inc, CA, USA). 3.3 and 7G8 hybridoma cells were also stained with biotinylated 4arm-PEG_{10K} (biotin-PEG, 0.5 nM) or biotinylated beta-glucuronidase (biotin-βG, 5 μg/mL) in HBSS, 2% FBS for 30 min at 4° C. followed by Alexa Fluor 647-conjugated streptavidin (2 μg/mL) (Jackson ImmunoResearch Laboratories, West Grove, Pa.) for 30 min at 4° C. Unbound probes were removed by washing with cold PBS twice. The surface fluorescence of 10⁴ viable cells was measured on a BD™ LSR II flow cytometer (Becton Dickinson, Mountain View, Calif., USA) and analyzed with Flowjo (Tree Star Inc., San Carlos, Calif., USA).

Lentiviral Transduction of AID Genes into Hybridomas

Recombinant lentiviral particles were packaged by co-transfection of 7.5 μg pCMV-AID-IoxP with 6.75 μg pCMVAR8.91 and 0.75 μg pMD.G using 45 μL TransIT-LT1 transfection reagent (Mirus Bio, Madison, Wis.) in 293FT cells grown in a 10 cm culture dish (90% confluency). After 48 h, lentiviral particles were harvested and concentrated by ultracentrifugation (Beckman SW 41 Ti Ultracentrifuge Swing Bucket Rotor, 50,000×g, 1.5 h, 4° C.). Lentiviral particles were suspended in culture medium containing 5 μg/mL polybrene and filtered through a 0.45 μm filter. 3.3 hybridoma cells were seeded in 6-well plates (1×10⁵ cells/well) one day before viral infection. Lentivirus containing medium was added to the cells, which were then centrifuged for 1.5 h (500×g, 32° C.). The cells were selected in complete medium containing puromycin (5 μg/mL) to generate stable 3.3/IoxP-AID, AG P4/IoxP-AID or 3 D8/IoxP-AID (pCMV-AID-IoxP) cells.

Conditional Expression of Exogenous mAID in Cells

The relative expression level of AID in cells was measured on a BD™ LSR II flow cytometer (Becton Dickinson, Mountain View, Calif., USA) by detection of the eGFP reporter in 3.3/IoxP-AID and 3T3/TetOn-AID cells in the presence or absence of doxycycline. To examine if AID expression could be stopped, 3.3/IoxP-AID hybridoma cells (2.5×10⁶ cells) were transfected with 5 μg of pLM-CMV-mCherry-P2A-Cre DNA in electroporation solution (Mirus Bio LLC, WI, USA) using a BTX electroporator (275 voltage, 15 msec pulse length). The cells were cultured in a 6-well plate for 48 hours and then analyzed for eGFP and mCherry fluorescence on a BD™ LSR II flow cytometer. pLM-CMV-mCherry-P2A-Cre transfected 3.3/IoxP-mAID cells that were negative for eGFP expression were isolated on a FACSAria cell sorter. To directly measure AID protein levels in cells, 5×10⁶ 3.3 or 3.3/IoxP-AID hybridoma cells were lysed in 0.5 mL RIPA buffer (1% NP-40, 150 mM NaCl, 0.5% sodium deoxycholate, 0.1% SDS, 50 mM Tris, pH 8.0) for 1 hour at 4° C. Fifty μg of total protein from the clarified lysate was electrophoresed on a 12.5% reducing SDS-PAGE, transferred to nitrocellulose paper and sequentially stained with biotinylated goat anti-HA (Vector Laboratories, Inc, CA, USA) or rabbit anti-tubulin alpha antibody (NeoMarkers Inc, CA, USA) followed by streptavidin-HRP and goat anti-rabbit Ig-HRP, respectively (Jackson ImmunoResearch Laboratories, Inc, PA, USA). Bands were visualized by ECL detection (Pierce, Rockford, Ill.) and analyzed with a LAS-3000 Mini Fujifilm imaging system (FujiFilm, Tokyo, Japan).

### Somatic Hypermutation Assay

3T3 or 3T3/TetOn-mAID cells were infected with sDsRed2 lentivirus to generate 3T3/sDsRed2 or 3T3/TetOn-mAID×sDsRed2 cells. The cells were cultured with or without 500 ng/mL doxycycline. Cells were harvested at defined times and processed for flow cytometry to measure the DsRed signal, indicative of mutation of the premature stop codon to allow expression of full length DsRed protein. The percentage of DsRed2-positive cells were calculated and reported as revertants/10⁶ cells.

### Isolation of Anti-PEG Antibody Variants by FACS

To isolate anti-PEG antibody variants, 3×10⁷ 3.3/IoxP-AID cells were stained with biotinylated 4arm-PEG_{10K} (100 pM, 1×10⁶ cells/mL) in HBSS, 2% FBS for 30 min at 4° C. followed by incubation for 30 min at 4° C. with 5 mL of Alexa Fluor 647-conjugated streptavidin (2 μg/mL) and PE-conjugated goat anti-mouse IgG Fc antibody (2 μg/mL) to measure membrane-bound immunoglobulin levels. Unbound probes were removed by washing with cold PBS twice. Cells displaying high Alexa Fluor 647 fluorescence (1% of total cells) were collected on a FACSAria cell sorter and cultured for 2 weeks before the cells were sorted again. The PEG chain length of the PEG probe was progressively decreased from 4arm-PEG_{10K}, linear PEG_{5K} and linear PEG_{2K} during subsequent rounds of sorting. After 5 rounds, single 3.3/mAID cells were collected into 96-well plates and AID expression was stopped by transient transfection of pLM-CMV-mCherry-P2A-Cre.

### Antibody Production and Purification

2.5×10⁷ of selected 3.3/IoxP-AID variant hybridoma cells (1E3 and 2B5) in 15 mL culture medium (DMEM, 5% FBS) were inoculated into a CELLine CL 1000 two-compartment bioreactor (INTEGRA Biosciences AG, Hudson, N.H.). The antibody-containing culture medium was harvested every 7 days and then purified by protein A Sepharose 4 Fast Flow chromatography (GE Healthcare, Piscataway, N.J.). Collected antibody was dialyzed against PBS and sterile filtered. Antibody concentrations were determined by the BCA protein assay (Thermo Scientific Pierce, Rockford, Ill.).

### Site Directed Mutagenesis of Antibody 2B5

The recombinant 2B5 antibody gene was cloned from 2B5 hybridoma cDNA by RT-PCR. The 2B5 light chain and heavy chain DNA were joined by a composite furin-2A bicistronic expression peptide linker in pLNCX-anti-PEG-eB7. An EcoR I-Pme I digested 2B5 IgG fragment was inserted into pAS3w.Ppuro to create pAS3w.Ppuro-2B5. Site-directed mutagenesis of V23A and K54N was carried out in a 50 μL mixture containing 20 ng of pAS3w.Ppuro-2B5 template DNA plasmid, 15 pmole of each primer, 20 nmole of dNTPs, 2 U of Phusion high-fidelity DNA polymerase (Thermo Scientific) in 1× Phusion buffer. Thermal cycling used an initial denaturation at 95° C. for 0.5 min; 18 cycles at 95° C. for 0.5 min, 55° C. for 1 min and 68° C. for 11 min. After cooling to ≦37° C., 2 U of Dpn I restriction enzyme (NEB, Beverly, Mass.) was directly added to the amplification reaction at 37° C. for 1.5 h. Four microliter of the Dpn I digested sample was used for the transformation of DH5a competent cells by the heat shock method. 3T3 cells that stably secreted 2B5/V23A (2B5 ΔV) and 2B5/K54N (2B5 ΔK) antibodies were generated by lentiviral transduction and selected in puromycin (10 μg/mL) as described above. The culture medium for 2B5/V23A and 2B5/K54N recombinant antibodies was harvested from CELLine adhere 1000 bioreactors every 7-10 days and the antibodies were purified by protein A Sepharose 4 Fast Flow chromatography.

### Antibody ELISA

Maxisorp 96-well microplates (Nalge-Nunc International, Roskilde, Denmark) were coated with 0.5 μg/well methoxy-PEG₇₅₀-NH₂, methoxy-PEG_{1K}-NH₂ methoxy-PEG_{2K}-NH₂, methoxy-PEG_{3K}-NH₂ hydroxy-PEG_{5K}-N H₂, methoxy-PEG_{10K}-NH₂, methoxy-PEG_{20K}-NH₂, and 4-arm poly(ethylene oxide)_{10K}-NH₂ in 50 μL/well 0.1 M NaHCO₃/Na₂CO₃ (adjusted to pH 8.0 with HCl) buffer for 3 h at 37° C. and then blocked with 200 μL/well dilution buffer (5% skim milk in PBS) at 4° C. overnight. Antibodies were pre-incubated at 37° C. for up to 5 days to check thermal stability. Graded concentrations of antibodies in 50 μL 2% skim milk were added to the plates at 4° C., RT or 37° C. for 1 h. The plates were washed with PBS three times at 4° C., RT or 37° C., respectively. HRP-conjugated donkey anti-mouse IgG Fc (2 μg/mL) in 50 μL dilution buffer were added for 1 h at 4° C., RT or 37° C. The plates were washed as described above. For competition ELISA assays, maxisorp 96-well microplates were coated with 0.5 μg/well amino-PEG_{3K}-NH₂, amino-PEG_{10K}-NH₂ or human beta-glucuronidase as described above. Three-fold serial dilutions of 18-crown-6 starting at 120 mM were prepared and mixed 1:1 (v/v) with 20 μg/mL of 3.3, 2B5 or 7G8 antibodies (thus the final concentration of the antibodies was 10 μg/mL). The mixture was added to the plates at 4° C. for 1 h. The plates were washed with PBS three times at 4° C. and were then incubated with HRP-conjugated donkey anti-mouse IgG Fc (2 μg/mL) at 4° C. for 1 h. The bound peroxidase activity was measured by adding 150 μL/well ABTS solution [0.4 mg/mL, 2,2′-azinobis(3-ethylbenzthiazoline-6-sulfonic acid), 0.003% H₂O₂, and 100 mM phosphate-citrate, pH 4.0) for 30 min at room temperature. The absorbance (405 nm) of wells was measured in a microplate reader (Molecular Device, Menlo Park, Calif.).

### Thermal Stability Analysis of Antibodies

Antibodies were dialyzed in PBS, degassed, and added into the sample chamber of a differential scanning calorimeter (Nano DSC III) (TA Instruments, New Castle, Del., USA) at concentrations of 0.5 mg/mL. Degassed PBS was injected into the reference chamber. Differential power was monitored as each antibody-buffer pair was heated linearly from 10° C. to 110° C. at a rate of 1° C. per minute under a fixed pressure of 3 atm. Buffer-buffer (degassed PBS) scans were also collected for baseline subtraction using the same procedure as for the antibody samples.

### Crown Ether Specific Binding Analysis of Antibodies by Surface Plasmon Resonance

The binding activity of antibodies to 18-crown-6 compounds was measured on a Biacore T-200 (GE Healthcare, Piscataway, N.J.) at defined temperatures. The 2-aminomethyl-18-crown-6 was immobilized on a CM5 chip by using the standard procedure for amine coupling through the EDC/NHS reaction. Injection of 2-aminomethyl-18-crown-6 (10 mM in 50 mM sodium borate buffer, pH 8.5) to a EDC/NHS activated CM5 chip was carried out at a constant flow rate of 10 μL/min for 30 minutes. The remaining succinimide esters were inactivated by the injection of ethanolamine. A total immobilization of 279.4 resonance units (RUs) was achieved. Antibody binding analysis was carried out at a constant flow rate of 50 μL/min of the antibodies at 1 μM in HEPES buffered saline at 4° C., 25° C. and 37° C.

### Affinity Purification of PEGylated Nanoparticles by Anti-PEG Antibodies

The PEG-Qdot 655 nanoparticles were purified by affinity chromatography on the anti-PEG antibody resin columns. Briefly, desalt 5 mg of anti-PEG antibodies to coupling buffer (0.1M sodium acetate, 0.15M sodium chloride, pH 5.5) to a final volume of 1 mL by using Zeba™ Spin Desalting Columns (Thermo Scientific Pierce, Rockford, Ill.). Add 2.1 mg of sodium meta-periodate (Thermo Scientific Pierce, Rockford, Ill.) to the antibody solution to a final concentration of 10 mM and incubate the mixture in the dark at room temperature for 30 minutes. Before incubating the oxidized antibodies with 1 mL of UltraLink® Hydrazide Resin containing 0.1M aniline (Thermo Scientific Pierce, Rockford, Ill.) at room temperature for 4 hours, sodium meta-periodate in the antibody solution was removed by Zeba™ Spin Desalting Columns. The anti-PEG antibodies coupled resin columns were washed with PBS for 3 times (2 mL/time). Five hundred microliter of PEG-Qdot 655 solution (32 nM) was loaded into the anti-PEG resin column at 4° C. then washed with cold PBS. Bound PEG-Qdot 655 nanoparticles were either eluted by 100 mM citrate buffer (pH=3) or heating PBS (37° C.).

### Immunoglobulin Isotyping ELISA

Antibody class and subclasses were determined by ELISA using a Mouse MonoAb-ID kit (Zymed Laboratories) according the suppliers instructions.

### FACS Analysis of Class Switched 3D8 Antibody Variants

B16F1 cells were stained with culture supernatant of 3D8 antibody variants for 30 min at 4° C. The cells were washed three times with cold PBS and stained with 2 μg/mL PE-conjugated goat Ig anti-mouse IgG Fc antibody (Jackson ImmunoResearch Laboratories, West Grove, Pa.). Unbound probes were removed by washing with cold PBS twice. The surface fluorescence of 10⁴ viable cells was measured on a BD™ LSR II flow cytometer (Becton Dickinson, Mountain View, Calif., USA) and analyzed with Flowjo (Tree Star Inc., San Carlos, Calif., USA).

### Production of FO-AID Cells

FO myeloma cells (ATCC PTA-11450) were infected with recombinant lentivirus that allows constitutive expression of AID under control of the CMV promoter (based on pCMV-AID-IoxP). The AID gene is flanked by IoxP sites to allow Cre-mediated excision to stop somatic hypermutation when desired. Stable cells were isolated by culture in medium supplemented with puromycin. Fluorescence-activated cell sorting of eGFP positive cells was performed to ensure all cells express AID. The expression of AID protein was confirmed by immunoblotting of cell lysates.

### Additional Methods

HMMA 2.5 cells is a heterohybridoma formed between mouse myeloma cell line P3X63Ag8.653 and bone marrow mononuclear cells from a patient suffering from IgA meyloma.⁷⁴ These cells will be stably infected with recombinant lentiviral particles that express an activation-induced cytidine deaminase gene under the control of a CMV promoter or in a Tet-on inducible vector. After selection in puromycin, cells that express eGFP, representative of AID expression, are collected by fluorescence-activated cell sporting. HMMA 2.5-AID cells are treated to ensure they are free of mycoplasma and then lots of cells will be banked in liquid nitrogen.

Human immune mice are established based on previously published methods.^{62,75} Briefly, umbilical cord blood is depleted of red blood cells followed by Percoll gradient centrifugation. CD34⁺ cells are isolated by magnetic bead isolation. Newborn (24-48 h old) NOD-scid IL2rγ^{null} (NSG) mice are irradiated with 100 cGy and then injected with 1×10⁵ CD34⁺ hematopoietic stem cells via intrahepatic injection directly through the skin. Human cell populations in engrafted mice are periodically analyzed by fluoresce-activated cell sorting using fluorescence-labeled antibodies against specific human markers of differentiation.

Reconstituted human immune mice are immunized by s.c. injection with antigens in complete Freund's adjuvant. The mice are then be boosted every three weeks with diminishing quantities of antigen in incomplete Freund's adjuvant. Blood samples, removed one week after each immunization, and are assayed for specific antibody titer by ELISA. Mouse hematopoietic cells are removed from splenocytes by binding to magnetic beads coated with rat anti-mouse CD45 antibody 30-F11. Human splenocytes are stimulated for transformation with the TLR agonists CpG 2006 and culture medium from persistently infected and transformed B95-8 cells which produce Epstein Barr virus as previously described.⁵⁶ HMMA2.5-AID and EBV-transformed human B cells are fused by electrofusion or PEG methods and cultured in complete RPMI-1640 medium supplemented with 20% heat-inactivated FBS, 2 mM L-glutamine, 1 mM sodium pyruvate, 2.5 μg/ml amphotericin, 50 μg/ml gentamicin, 60 μg/ml tylosin solution, 1×HAT (100 μM hypoxanthine, 0.4 μM aminopterin, 16 μM thymidine), 5 μg/ml puromycin and 0.5 μM ouabain. Ouabain is used to eliminate non-fused EBV-transformed B cells, puromycin can eliminate hybridoma cells that don't express AID and HAT is used to eliminate unfused myeloma cells. After culture for one week, the medium is slowly changed to include HT (100 μM hypoxanthine, 16 μM thymidine) rather than HAT. Positive hybridomas can be identified by measuring specific antibody in culture medium by ELISA.

For example, to screen for human monoclonal antibodies against neuropilin-1 (NRP-1), human hybridoma cells are stained with the biotinylated b1b2 domain of NRP-1 followed by incubation with Alexa647-conjugated streptavidin and PE-conjugated goat anti-human Ig (A+G+M) antibody to measure membrane-bound immunoglobulin levels. Cells displaying Alexa647 fluorescence are collected on a FACSAria cell sorter. To identify hybridoma cells that exhibit functional neutralization of NRP-1, the binding of the b1b2 domain of NRP-1 is competed for in the presence of Alexa488-conjugated VEGF-A₁₆₅. Cells displaying high Alexa647 fluorescence (representing hybridoma cells that express anti-NRP-1 antibody and therefore can bind biotinylated NRP-1 and Alexa647-conjugated streptavidin) without Alexa488 fluorescence (representing hybridoma cells in which the anti-NRP-1 antibody blocks binding of Alexa488-conjugated VEGF-A₁₆₅) are collected on a FACSAria cell sorter. To analyze the functional blockage of NRP-1 antibodies, dextran-coated Cytodex 3 microcarrier beads (Amersham) are incubated with HUVECs in the presence of VEGF-A₁₆₅, and antibodies are added to each well. Eight days later the beads are imaged on an inverted microscope. The sprout length and number of vessels is counted to identify monoclonal antibodies with the greatest antiangiogenesis activity. The protein concentration of Alexa488-conjugated VEGF-A₁₆₅ is progressively increased during subsequent rounds of sorting. In the final cell selection, single anti-NRP-1 hybridoma cells are collected into 96-well plates and AID expression is stopped by transient transfection of a pLM-CMV-mCherry-P2A-Cre DNA plasmid expressing Cre recombinase.

### Prior Art

Several antibody display libraries have been developed for screening high affinity antibodies, including phage,⁴⁴ yeast⁸ and ribosome⁴⁵ display. These technologies only allow non-glycosylated antibody fragment display but not full-length IgGs with proper glycosylation. In addition, substantial library development and molecular cloning is required.

A human antibody display library has been generated from human donors. This library was expressed on mammalian cells. This method is limited to the discovery of antibodies against non-human antigens from patients, such as Qβ virus like particle (VLP), a model viral antigen.⁴⁶

The expression of activation-induced cytidine deaminase (AID) is sufficient to induce somatic hypermutation as well as class switch recombination in hybridomas and fibroblasts.^{38, 47-49} No attempt to select high affinity antibodies was described in these publications.

Stable transfection of activation-induced cytidine deaminase (AID) in hybridomas was demonstrated to increase the frequency of class switching to facilitate the isolation of subclones expressing monoclonal antibodies of different isotypes, but this technology was not used to isolate high affinity antibodies.³⁷

A monomeric red fluorescent protein gene was stably expressed in the AID over-expressing Ramos cells (Human Burkitt's lymphoma cell line) where it can be evolved into a red fluorescent proteins with increased photostability and far-red emissions (e.g., 649 nm) by AID mediated SHM. Therefore, SHM offers a strategy to evolve nonantibody proteins with desirable properties. This system might be applied to antibody affinity maturation by transfecting Ramos cells with the desired antibody genes. However, it is time-consuming for target gene transfection, iterative isolation (about 23 rounds) and protein expression.⁵⁰

The AID overexpressing B cell lines (human Ramos and chicken DT-40) display surface IgM allowing isolation of antigen-specific B cells which recognized fluorescently labeled antigens on the B cell membrane by using FACS after AID dependent SHM. Although Ramos cells produce human antibodies, the iterative evolution and selection procedure is not efficient (about 19 rounds, 2 weeks/round).⁵¹

B cell lymphoma-6 (Bcl-6) and Bcl-xL genes were introduced into peripheral blood memory B cells. Culture of these cells with CD40 ligand (CD40L) and interleukin-21 (IL-21) proteins result in highly proliferating, cell surface B cell receptor (BCR)-positive, immunoglobulin-secreting B cells with features of germinal center B cells, including expression of activation-induced cytidine deaminase (AID). However, this method is limited to the discovery of antibodies against non-human antigens since patients need to be immunized prior to B cell isolation.⁵²

Mammalian cell surface human antibody display with AID expression in human 293 cells has been applied to antibody affinity maturation. This technology successfully increased the affinity of an antibody via AID-dependent SHM by introducing an AID gene to the 293 cells. However, construction of the antibody library, AID gene introduction, re-transfection of isolated antibody genes and antibody expression steps are expensive, technologically-challenging and time-consuming.⁵³

### REFERENCES

1. Wu, H., Beuerlein, G., Nie, Y., Smith, H., Lee, B. A., Hensler, M., Huse, W. D., and Watkins, J. D. (1998) Stepwise in vitro affinity maturation of Vitaxin, an alphav beta3-specific humanized mAb, Proc Natl Acad Sci USA 95, 6037-6042.
2. Burtrum, D., Zhu, Z., Lu, D., Anderson, D. M., Prewett, M., Pereira, D. S., Bassi, R., Abdullah, R., Hooper, A. T., Koo, H., Jimenez, X., Johnson, D., Apblett, R., Kussie, P., Bohlen, P., Witte, L., Hicklin, D. J., and Ludwig, D. L. (2003) A fully human monoclonal antibody to the insulin-like growth factor I receptor blocks ligand-dependent signaling and inhibits human tumor growth in vivo, Cancer Res 63, 8912-8921.
3. Lonberg, N., Taylor, L. D., Harding, F. A., Trounstine, M., Higgins, K. M., Schramm, S. R., Kuo, C. C., Mashayekh, R., Wymore, K., McCabe, J. G., and et al. (1994) Antigen-specific human antibodies from mice comprising four distinct genetic modifications, Nature 368, 856-859.
4. Green, L. L., Hardy, M. C., Maynard-Currie, C. E., Tsuda, H., Louie, D. M., Mendez, M. J., Abderrahim, H., Noguchi, M., Smith, D. H., Zeng, Y., David, N. E., Sasai, H., Garza, D., Brenner, D. G., Hales, J. F., McGuinness, R. P., Capon, D. J., Klapholz, S., and Jakobovits, A. (1994) Antigen-specific human monoclonal antibodies from mice engineered with human Ig heavy and light chain YACs, Nat Genet 7, 13-21.
5. Lonberg, N. (2005) Human antibodies from transgenic animals, Nat Biotechnol 23, 1117-1125.
6. Kuroiwa, Y., Kasinathan, P., Sathiyaseelan, T., Jiao, J. A., Matsushita, H., Sathiyaseelan, J., Wu, H., Mellquist, J., Hammitt, M., Koster, J., Kamoda, S., Tachibana, K., Ishida, I., and Robl, J. M. (2009) Antigen-specific human polyclonal antibodies from hyperimmunized cattle, Nat Biotechnol 27, 173-181.
7. Boder, E. T., and Wittrup, K. D. (1997) Yeast surface display for screening combinatorial polypeptide libraries, Nat Biotechnol 15, 553-557.
8. Feldhaus, M. J., Siegel, R. W., Opresko, L. K., Coleman, J. R., Feldhaus, J. M., Yeung, Y. A., Cochran, J. R., Heinzelman, P., Colby, D., Swers, J., Graff, C., Wiley, H. S., and Wittrup, K. D. (2003) Flow-cytometric isolation of human antibodies from a nonimmune Saccharomyces cerevisiae surface display library, Nat Biotechnol 21, 163-170.
9. Hoogenboom, H. R. (2005) Selecting and screening recombinant antibody libraries, Nat Biotechnol 23, 1105-1116.
10. Mazor, Y., Van Blarcom, T., Mabry, R., Iverson, B. L., and Georgiou, G. (2007) Isolation of engineered, full-length antibodies from libraries expressed in Escherichia coli, Nat Biotechnol 25, 563-565.
11, Wrammert, J., Smith, K., Miller, J., Langley, W. A., Kokko, K., Larsen, C., Zheng, N. Y., Mays, I., Garman, L., Helms, C., James, J., Air, G. M., Capra, J. D., Ahmed, R., and Wilson, P. C. (2008) Rapid cloning of high-affinity human monoclonal antibodies against influenza virus, Nature 453, 667-671.
12. Jin, A., Ozawa, T., Tajiri, K., Obata, T., Kondo, S., Kinoshita, K., Kadowaki, S., Takahashi, K., Sugiyama, T., Kishi, H., and Muraguchi, A. (2009) A rapid and efficient single-cell manipulation method for screening antigen-specific antibody-secreting cells from human peripheral blood, Nat Med 15, 1088-1092.
13. Iizuka, A., Komiyama, M., Tai, S., Oshita, C., Kurusu, A., Kume, A., Ozawa, K., Nakamura, Y., Ashizawa, T., Yamamoto, A., Yamazaki, N., Yoshikawa, S., Kiyohara, Y., Yamaguchi, K., and Akiyama, Y. (2011) Identification of cytomegalovirus (CMV)pp65 antigen-specific human monoclonal antibodies using single B cell-based antibody gene cloning from melanoma patients, Immunol Lett 135, 64-73.
14. Hu, J., Huang, X., Ling, C. C., Bundle, D. R., and Cheung, N. K. (2009) Reducing epitope spread during affinity maturation of an anti-ganglioside GD2 antibody, J Immunol 183, 5748-5755.
15. Brooks, C. L., Schietinger, A., Borisova, S. N., Kufer, P., Okon, M., Hirama, T., Mackenzie, C. R., Wang, L. X., Schreiber, H., and Evans, S. V. (2010) Antibody recognition of a unique tumor-specific glycopeptide antigen, Proc Acad Sci USA 107, 10056-10061.
16. Su, Y. C., Chen, B. M., Chuang, K. H., Cheng, T. L., and Roffler, S. R. (2010) Sensitive quantification of PEGylated compounds by second-generation anti-poly(ethylene glycol) monoclonal antibodies, Bioconjug Chem 21, 1264-1270.
17. Schier, R., McCall, A., Adams, G. P., Marshall, K. W., Merritt, H., Yim, M., Crawford, R. S., Weiner, L. M., Marks, C., and Marks, J. D. (1996) Isolation of picomolar affinity anti-c-erbB-2 single-chain Fv by molecular evolution of the complementarity determining regions in the center of the antibody binding site, J Mol Biol 263, 551-567.
18. Pini, A., Viti, F., Santucci, A., Carnemolla, B., Zardi, L., Neri, P., and Neri, D. (1998) Design and use of a phage display library. Human antibodies with subnanomolar affinity against a marker of angiogenesis eluted from a two-dimensional gel, J Biol Chem 273, 21769-21776.
19. Blaise, L., Wehnert, A., Steukers, M. P., van den Beucken, T., Hoogenboom, H. R., and Hufton, S. E. (2004) Construction and diversification of yeast cell surface displayed libraries by yeast mating: application to the affinity maturation of Fab antibody fragments, Gene 342, 211-218.
20. Wu, H., Pfarr, D. S., Tang, Y., An, L. L., Patel, N. K., Watkins, J. D., Huse, W. D., Kiener, P. A., and Young, J. F. (2005) Ultra-potent antibodies against respiratory syncytial virus: effects of binding kinetics and binding valence on viral neutralization, J Mol Biol 350, 126-144.
21. Hoet, R. M., Cohen, E. H., Kent, R. B., Rookey, K., Schoonbroodt, S., Hogan, S., Rem, L., Frans, N., Daukandt, M., Pieters, H., van Hegelsom, R., Neer, N. C., Nastri, H. G., Rondon, I. J., Leeds, J. A., Hufton, S. E., Huang, L., Kashin, I., Devlin, M., Kuang, G., Steukers, M., Viswanathan, M., Nixon, A. E., Sexton, D. J., Hoogenboom, H. R., and Ladner, R. C. (2005) Generation of high-affinity human antibodies by combining donor-derived and synthetic complementarity-determining-region diversity, Nat Biotechnol 23, 344-348.
22. van den Beucken, T., Pieters, H., Steukers, M., van der Vaart, M., Ladner, R. C., Hoogenboom, H. R., and Hufton, S. E. (2003) Affinity maturation of Fab antibody fragments by fluorescent-activated cell sorting of yeast-displayed libraries, FEBS Lett 546, 288-294.
23. Lu, D., Shen, J., Vil, M. D., Zhang, H., Jimenez, X., Bohlen, P., Witte, L., and Zhu, Z. (2003) Tailoring in vitro selection for a picomolar affinity human antibody directed against vascular endothelial growth factor receptor 2 for enhanced neutralizing activity, J Biol Chem 278, 43496-43507.
24. Zahnd, C., Spinelli, S., Luginbuhl, B., Amstutz, P., Cambillau, C., and Pluckthun, A. (2004) Directed in vitro evolution and crystallographic analysis of a peptide-binding single chain antibody fragment (scFv) with low picomolar affinity, J Biol Chem 279, 18870-18877.
25. Rothe, A., Hosse, R. J., and Power, B. E. (2006) Ribosome display for improved biotherapeutic molecules, Expert Opin Biol Ther 6, 177-187.
26. Corti, D., Voss, J., Gamblin, S. J., Codoni, G., Macagno, A., Jarrossay, D., Vachieri, S. G., Pinna, D., Minola, A., and Vanzetta, F. (2011) A neutralizing antibody selected from plasma cells that binds to group 1 and group 2 influenza A hemagglutinins, Science 333, 850.
27. Ekiert, D. C., Friesen, R. H. E., Bhabha, G., Kwaks, T., Jongeneelen, M., Yu, W., Ophorst, C., Cox, F., Korse, H. J. W. M., Brandenburg, B., Vogels, R., Brakenhoff, J. P. J., Kompier, R., Koldijk, M. H., Cornelissen, L. A. H. M., Poon, L. L. M., Peiris, M., Koudstaal, W., Wilson, I. A., and Goudsmit, J. (2011) A Highly Conserved Neutralizing Epitope on Group 2 Influenza A Viruses, Science 333, 843-850.
28. Ramsden, D. A., van Gent, D. C., and Gellert, M. (1997) Specificity in V(D)J recombination: new lessons from biochemistry and genetics, Curr Opin Immunol 9, 114-120.
29. Nagaoka, H., Muramatsu, M., Yamamura, N., Kinoshita, K., and Honjo, T. (2002) Activation-induced deaminase (AID)-directed hypermutation in the immunoglobulin Smu region: implication of AID involvement in a common step of class switch recombination and somatic hypermutation, J Exp Med 195, 529-534.
30. Durandy, A. (2003) Activation-induced cytidine deaminase: a dual role in class-switch recombination and somatic hypermutation, Eur J Immunol 33, 2069-2073.
31. Longerich, S., Basu, U., Alt, F., and Storb, U. (2006) AID in somatic hypermutation and class switch recombination, Curr Opin Immunol 18, 164-174.
32. Xu, Z., Pone, E. J., Al-Qahtani, A., Park, S. R., Zan, H., and Casali, P. (2007) Regulation of aicda expression and AID activity: relevance to somatic hypermutation and class switch DNA recombination, Crit Rev Immunol 27, 367-397.
33. Jacob, J., Przylepa, J., Miller, C., and Kelsoe, G. (1993) In situ studies of the primary immune response to (4-hydroxy-3-nitrophenyl)acetyl. III. The kinetics of V region mutation and selection in germinal center B cells, J Exp Med 178, 1293-1307.
34. Gonda, H., Sugai, M., Nambu, Y., Katakai, T., Agata, Y., Mori, K. J., Yokota, Y., and Shimizu, A. (2003) The balance between Pax5 and Id2 activities is the key to AID gene expression, J Exp Med 198, 1427-1437.
35. Crouch, E. E., Li, Z., Takizawa, M., Fichtner-Feigl, S., Gourzi, P., Montano, C., Feigenbaum, L., Wilson, P., Janz, S., Papavasiliou, F. N., and Casellas, R. (2007) Regulation of AID expression in the immune response, J Exp Med 204, 1145-1156.
36. Muto, A., Ochiai, K., Kimura, Y., Itoh-Nakadai, A., Calame, K. L., Ikebe, D., Tashiro, S., and Igarashi, K. (2010) Bach2 represses plasma cell gene regulatory network in B cells to promote antibody class switch, EMBO J 29, 4048-4061.
37. Iglesias-Ussel, M. D., Fan, M., Li, Z., Martin, A., and Scharff, M. D. (2006) Forced expression of AID facilitates the isolation of class switch variants from hybridoma cells, J Immunol Methods 316, 59-66.
38. Martin, A., Bardwell, P. D., Woo, C. J., Fan, M., Shulman, M. J., and Scharff, M. D. (2002) Activation-induced cytidine deaminase turns on somatic hypermutation in hybridomas, Nature 415, 802-806.
39. Chen, K. C., Cheng, T. L., Leu, Y. L., Prijovich, Z. M., Chuang, C. H., Chen, B. M., and Roffler, S. R. (2007) Membrane-localized activation of glucuronide prodrugs by beta-glucuronidase enzymes, Cancer Gene Ther 14, 187-200.
40. Chuang, K. H., Wang, H. E., Cheng, T. C., Tzou, S. C., Tseng, W. L., Hung, W. C., Tai, M. H., Chang, T. K., Roffler, S. R., and Cheng, T. L. (2010) Development of a universal anti-polyethylene glycol reporter gene for noninvasive imaging of PEGylated probes, J Nucl Med 51, 933-941.
41. Sawano, A., and Miyawaki, A. (2000) Directed evolution of green fluorescent protein by a new versatile PCR strategy for site-directed and semi-random mutagenesis, Nucleic Acids Res 28, E78.
42. Centlivre, M., Zhou, X., Pouw, S. M., Weijer, K., Kleibeuker, W., Das, A. T., Blom, B., Seppen, J., Berkhout, B., and Legrand, N. (2010) Autoregulatory lentiviral vectors allow multiple cycles of doxycycline-inducible gene expression in human hematopoietic cells in vivo, Gene Ther 17, 14-25.
43. Klasen, M., Spillmann, F. J., Lorens, J. B., and Wabl, M. (2005) Retroviral vectors to monitor somatic hypermutation, J Immunol Methods 300, 47-62.
44. Vaughan, T. J., Williams, A. J., Pritchard, K., Osbourn, J. K., Pope, A. R., Earnshaw, J. C., McCafferty, J., Hodits, R. A., Wilton, J., and Johnson, K. S. (1996) Human antibodies with sub-nanomolar affinities isolated from a large non-immunized phage display library, Nat Biotechnol 14, 309-314.
45. Hanes, J., Schaffitzel, C., Knappik, A., and Pluckthun, A. (2000) Picomolar affinity antibodies from a fully synthetic naive library selected and evolved by ribosome display, Nat Biotechnol 18, 1287-1292.
46. Beerli, R. R., Bauer, M., Buser, R. B., Gwerder, M., Muntwiler, S., Maurer, P., Saudan, P., and Bachmann, M. F. (2008) Isolation of human monoclonal antibodies by mammalian cell display, Proc Natl Acad Sci USA 105, 14336-14341.
47. Martin, A., and Scharff, M. D. (2002) Somatic hypermutation of the AID transgene in B and non-B cells, Proc Natl Acad Sci USA 99, 12304-12308.
48. Okazaki, I. M., Kinoshita, K., Muramatsu, M., Yoshikawa, K., and Honjo, T. (2002) The AID enzyme induces class switch recombination in fibroblasts, Nature 416, 340-345.
49. Yoshikawa, K., Okazaki, I. M., Eto, T., Kinoshita, K., Muramatsu, M., Nagaoka, H., and Honjo, T. (2002) AID enzyme-induced hypermutation in an actively transcribed gene in fibroblasts, Science 296, 2033-2036.
50. Wang, L., Jackson, W. C., Steinbach, P. A., and Tsien, R. Y. (2004) Evolution of new nonantibody proteins via iterative somatic hypermutation, Proc Natl Acad Sci USA 101, 16745-16749.
51. Cumbers, S. J., Williams, G. T., Davies, S. L., Grenfell, R. L., Takeda, S., Batista, F. D., Sale, J. E., and Neuberger, M. S. (2002) Generation and iterative affinity maturation of antibodies in vitro using hypermutating B-cell lines, Nat Biotechnol 20, 1129-1134.
52. Kwakkenbos, M. J., Diehl, S. A., Yasuda, E., Bakker, A. Q., van Geelen, C. M., Lukens, M. V., van Bleek, G. M., Widjojoatmodjo, M. N., Bogers, W. M., Mei, H., Radbruch, A., Scheeren, F. A., Spits, H., and Beaumont, T. (2010) Generation of stable monoclonal antibody-producing B cell receptor-positive human memory B cells by genetic programming, Nat Med 16, 123-128.
53. Bowers, P. M., Horlick, R. A., Neben, T. Y., Toobian, R. M., Tomlinson, G. L., Dalton, J. L., Jones, H. A., Chen, A., Altobell, L., 3rd, Zhang, X., Macomber, J. L., Krapf, I. P., Wu, B. F., McConnell, A., Chau, B., Holland, T., Berkebile, A. D., Neben, S. S., Boyle, W. J., and King, D. J. (2011) Coupling mammalian cell surface display with somatic hypermutation for the discovery and maturation of human antibodies, Proc Natl Acad Sci USA 108, 20455-20460.
54. Daugherty, A. L. and R. J. Mrsny (2006) Formulation and delivery issues for monoclonal antibody therapeutics. Adv. Drug Deliv. Rev., 58, 686-706.
55. Lowe, D., K. Dudgeon, R. Rouet, et al. (2011) Aggregation, stability, and formulation of human antibody therapeutics. Adv. Protein Chem. Struct. Biol., 84, 41-61.
56. Traggiai, E., S. Becker, K. Subbarao, et al. (2004) An efficient method to make human monoclonal antibodies from memory B cells: potent neutralization of SARS coronavirus. Nat. Med., 10, 871-75.
57. Simmons, C. P., N. L. Bernasconi, A. L. Suguitan, et al. (2007) Prophylactic and therapeutic efficacy of human monoclonal antibodies against H5N1 influenza. PLoS Med., 4, e178.
58, Yu, X., T. Tsibane, P. A. McGraw, et al. (2008) Neutralizing antibodies derived from the B cells of 1918 influenza pandemic survivors. Nature, 455, 532-36.
59. Beerli, R. R. and C. Rader (2010) Mining human antibody repertoires. MAbs, 2.
60. Wilson, P. C. and S. F. Andrews (2012) Tools to therapeutically harness the human antibody response. Nat. Rev. Immunol., 12, 709-19.
61. Choi, B., E. Chun, M. Kim, et al. (2011) Human T cell development in the liver of humanized NOD/SCID/IL-2Rgamma(null)(NSG) mice generated by intrahepatic injection of CD34(+) human (h) cord blood (CB) cells. Clin Immunol., 139, 321-35.
62. Brehm, M. A., A. Cuthbert, C. Yang, et al. (2010) Parameters for establishing humanized mouse models to study human immunity: analysis of human hematopoietic stem cell engraftment in three immunodeficient strains of mice bearing the IL2rgamma(null) mutation. Clin. Immunol., 135, 84-98.
63. Onoe, T., H. Kalscheuer, N. Danzl, et al. (2011) Human natural regulatory T cell development, suppressive function, and postthymic maturation in a humanized mouse model. J. Immunol., 187, 3895-903.
64. Shultz, L. D., M. A. Brehm, J. V. Garcia-Martinez, et al. (2012) Humanized mice for immune system investigation: progress, promise and challenges. Nat. Rev. Immunol., 12, 786-98.
65. Traggiai, E., L. Chicha, L. Mazzucchelli, et al. (2004) Development of a human adaptive immune system in cord blood cell-transplanted mice. Science, 304, 104-07.
66. Tonomura, N., K. Habiro, A. Shimizu, et al. (2008) Antigen-specific human T-cell responses and T cell-dependent production of human antibodies in a humanized mouse model. Blood, 111, 4293-96.
67. De Giovanni, C., G. Nicoletti, L. Landuzzi, et al. (2012) Human responses against HER-2-positive cancer cells in human immune system-engrafted mice. Br. J. Cancer, 107, 1302-09.
68. Becker, P. D., N. Legrand, C. M. van Geelen, et al. (2010) Generation of human antigen-specific monoclonal IgM antibodies using vaccinated “human immune system” mice. PLoS One, 5.
69. Ippolito, G. C., K. H. Hoi, S. T. Reddy, et al. (2012) Antibody repertoires in humanized NOD-scid-IL2Rgamma(null) mice and human B cells reveals human-like diversification and tolerance checkpoints in the mouse. PLoS One, 7, e35497.
70. Kaveri, S. V., G. J. Silverman, and J. Bayry (2012) Natural IgM in immune equilibrium and harnessing their therapeutic potential. J. Immunol., 188, 939-45.
71. Vuyyuru, R., J. Patton, and T. Manser (2011) Human immune system mice: current potential and limitations for translational research on human antibody responses. Immunol. Res., 51, 257-66.
72. Schmidt, M. R., M. C. Appel, L. J. Giassi, et al. (2008) Human BLyS facilitates engraftment of human PBL derived B cells in immunodeficient mice. PLoS One, 3, e3192.
73. Suzuki, M., T. Takahashi, I. Katano, et al. (2012) Induction of human humoral immune responses in a novel HLA-DR-expressing transgenic NOD/Shi-scid/gammacnull mouse. Int. Immunol., 24, 243-52.
74. Lang, J., M. Kelly, B. M. Freed, et al. (2013) Studies of lymphocyte reconstitution in a humanized mouse model reveal a requirement of T cells for human B cell maturation. J. Immunol., 190, 2090-101.
75. Posner, M. R., H. Elboim, and D. Santos (1987) The construction and use of a human-mouse myeloma analogue suitable for the routine production of hybridomas secreting human monoclonal antibodies. Hybridoma, 6, 611-25.
76. Choi, B., E. Chun, M. Kim, et al. (2011) Human B cell development and antibody production in humanized NOD/SCID/IL-2Rgamma(null) (NSG) mice conditioned by busulfan. J. Clin. Immunol., 31, 253-64.

We claim:

1. A process of affinity maturation in existing monoclonal-antibody producing hybridoma cells, said process comprising:
(a) expressing activation-induced cytidine deaminase in said existing hybridoma cells using a controllable activation-induced cytidine deaminase expression system;
(b) determining both expression levels and antigen-binding ability of antibodies naturally present on the surface of said existing hybridoma cells; and
(c) selecting said hybridoma cells that secrete high affinity antibodies by fluorescence-activated cell sorting of said hybridoma cells that preferentially bind fluorescence-labeled antigen via said antibodies that are naturally present on the surface of said hybridoma cells.

2. The process of claim 1, wherein the process is repeated until hybridomas secreting sufficiently high affinity antibodies are isolated.

3. The process of claim 2, wherein step (a) is terminated by transfection of the cells with a CRE recombinase.

4. The process of claim 2, wherein step (a) is terminated by removal of doxycycline.

5. The process of claim 1, wherein said antibodies undergo class switch recombination.

6. The process of claim 5, wherein said antibodies undergo class switch recombination from IgM to a class selected from the group consisting of IgG, IgA, and IgE.

7. The process of claim 1, wherein said hybridoma cells are hybridoma cells that secrete fully human antibody.

8. The process of claim 5, wherein said class switch recombination occurs in hydridoma cells that secrete fully human antibody.

9. The process of claim 6, wherein said class switch recombination occurs in hydridoma cells that secrete fully human antibody.

10. A process for generating new monoclonal antibody producing hybridoma cells with the ability to undergo tunable antibody affinity maturation, said process comprising:
(a) expressing activation-induced cytidine deaminase in myeloma hybridoma fusion partner cells using a controllable activation-induced cytidine deaminase expression system;
(b) generating hybridoma cells between the said myeloma cells expressing activation-induced cytidine deaminase and B cells from animals immunized with antigen; and
(c) selecting said hybridoma cells that secrete high affinity antibodies by fluorescence-activated cell sorting of said new hybridoma cells that preferentially bind fluorescence-labeled antigen via said antibodies that are naturally present on the surface of said hybridoma cells.

11. The process of claim 10, wherein the process is repeated until hybridomas secreting sufficiently high affinity antibodies are isolated.

12. The process of claim 11, wherein step (a) is terminated by transfection of the cells with a CRE recombinase.

13. The process of claim 11, wherein step (a) is terminated by removal of doxycycline.

14. The process of claim 10, wherein said antibodies undergo class switch recombination.

15. The process of claim 14, wherein said antibodies undergo class switch recombination from IgM to a class selected from the group consisting of IgG, IgA, and IgE.

16. The process of claim 10, wherein said hybridoma cells secrete fully human antibody.

17. The process of claim 14, wherein said class switch recombination occurs in hydridoma cells that secrete fully human antibody.

18. The process of claim 15, wherein said class switch recombination occurs in hydridoma cells that secrete fully human antibody.


SEQUENCE LISTING (8 sequences)

SEQ ID NO: 1 - protein, 40 aa, Artificial Sequence
  Hybridoma
EVKLVESGGGLVQPGGSMKLSCAASGFIFSDAWMDWVRQS

SEQ ID NO: 2 - protein, 40 aa, Artificial Sequence
  Hybridoma
EVKLVESGGGLVQPGGSMKLSCVASGFIFSDAWMDWVRQS

SEQ ID NO: 3 - protein, 10 aa, Artificial Sequence
  Hybridoma
SASSSVRSSY

SEQ ID NO: 4 - protein, 7 aa, Artificial Sequence
  Hybridoma
STSNLAS

SEQ ID NO: 5 - protein, 7 aa, Artificial Sequence
  Hybridoma
STSKLAS

SEQ ID NO: 6 - protein, 7 aa, Artificial Sequence
  Hybridoma
STSKLPS

SEQ ID NO: 7 - protein, 9 aa, Artificial Sequence
  Hybridoma
HQWSSYPRT

SEQ ID NO: 8 - protein, 9 aa, Artificial Sequence
  Hybridoma
HQWSTYPRT